SEQUENCE LISTING (38 sequences)

SEQ ID NO: 1 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDKKGTEAAGAMFLEAIPMSIPPEVKFNKPFVFLMIEQNTKSPLFMGKVVNPTQK

SEQ ID NO: 2 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 3 - protein, 8 aa, Mus musculus
QDINSYLS

SEQ ID NO: 4 - protein, 11 aa, Mus musculus
TLIYRANRLVD

SEQ ID NO: 5 - protein, 8 aa, Mus musculus
LQYDEFPY

SEQ ID NO: 6 - protein, 10 aa, Mus musculus
GYIFTSYWMN

SEQ ID NO: 7 - protein, 18 aa, Mus musculus
WIGKIDPSDSETHYNQKF

SEQ ID NO: 8 - protein, 10 aa, Mus musculus
VRSPGGYPDW

SEQ ID NO: 9 - protein, 161 aa, Mus musculus
RTPAQFLGILLLWFPGIKCDITMTQSPSSIYASLGKRVTITCKASQDINSYLSWFQQKPGKSPKTLIYRANRLVDGVPSRFSGSGSGQDYSLTISSLEYEDMGIYYCLQYDEFPYTFGGGTKLEIKRADAAPTVSIFPPSSEQLTSGGASVVCFLNNFYPK

SEQ ID NO: 10 - protein, 197 aa, Mus musculus
MTSLFSLQLLSTQDLAMGWSCIILFLAATATGVHSQVQLQQPGAELVKPGAPVKLSCKASGYIFTSYWMNWVKQRPGRGLEWIGKIDPSDSETHYNQKFKDKATLTVDKSSSTAFIQLSSLTSEDSAVYYCVRSPGGYPDWWGQGTTLTVSSAKTTPPSVYPLAPGSAAQTNSMVTLGCLVKGYFPEPVTVTWNSGS

SEQ ID NO: 11 - protein, 8 aa, Mus musculus
QDVGTAVA

SEQ ID NO: 12 - protein, 11 aa, Mus musculus
LLIYWASTRHT

SEQ ID NO: 13 - protein, 8 aa, Mus musculus
QQYSTYPL

SEQ ID NO: 14 - protein, 10 aa, Mus musculus
GYTFTSYWIT

SEQ ID NO: 15 - protein, 18 aa, Mus musculus
WIGDIYPGSGSTNYNEKF

SEQ ID NO: 16 - protein, 9 aa, Mus musculus
AAAVKGFAY

SEQ ID NO: 17 - protein, 160 aa, Mus musculus
ETHSQVFVYMLLLSGVEGDIVMTQSHKFMSTSVGDRVSITCKASQDVGTAVAWYQQKPGQSPKLLIYWASTRHTGVPDRFTGSGSGTDFTLTISNVQSEDLADYFCQQYSTYPLTFGSGTKLEIKRADAAPTVSIFPPSSEQLTSGGASVVCFLNNFYPK

SEQ ID NO: 18 - protein, 195 aa, Mus musculus
IRSLSLQLLSTQDLTMGWSCIILILVAAATGVHSQVQLQQPGAELVKPGASVKMSCKASGYTFTSYWITWVKQRPGQGLEWIGDIYPGSGSTNYNEKFKNKATLTVDTSSSTAYMQLSSLTSEDSAVYYCAAAVKGFAYWGQGTLVTVSAAKTTPPSVYPLAPGSAAGTNSMVTLGCLVKGYFPEPVTVTWNSGS

SEQ ID NO: 19 - protein, 158 aa, Mus musculus
ETHSQVFVYMLLSGVEGDIVMTQSHKFMSTSVGDRVSITCKASQDVGTAVAWYQQKPGQSPKLLIYWASTRHTGVPDRFTGSGSGTDFTLTITNVQSENLADYFCQQYSTYPLTFGSGTKLEIKRADAAPTVSIFPPSSEQLTSGGASVVCFLNNFYP

SEQ ID NO: 20 - protein, 196 aa, Mus musculus
IRSLFSLQLLSTQDLTMGWSCIILILVAAATGVHSQVQLQQPGAELVKPGASVKMSCKASGYTFTSYWITWVKQRPGQGLEWIGDIYPGSGSTNYNEKFKNKATLTVDTSSSTAYMQLSSLTSEDSAVYYCAAAVKGFAYWGQGTLVTVSAAKTTPPSVYPLAPGSAAQTNSMVTLGCLVKGYFPEPVTVTWNSGS

SEQ ID NO: 21 - protein, 161 aa, Mus musculus
ETHSQVFVYMLLWLSGVEGDIVMTQSHKFMSTSVGDRVSITCKASQDVGTAVAWYQQKPGQSPKLLIYWASTRHTGVPDRFTGSGSGTDFTLTISNVQSEDLADYFCQQYSTYPLTFGSGTKLEIKRADAAPTVSIFPPSSEQLTSGGASVVCFLNNFYPK

SEQ ID NO: 22 - protein, 196 aa, Mus musculus; identical to SEQ ID NO: 20

SEQ ID NO: 23 - protein, 160 aa, Mus musculus; identical to SEQ ID NO: 17

SEQ ID NO: 24 - protein, 196 aa, Mus musculus; identical to SEQ ID NO: 20

SEQ ID NO: 25 - protein, 162 aa, Mus musculus
ETHSQVFVYMLLLSGVEGDIVMTQSHKFMSTSVGDRVSITCKASQDVGTAVAWYQQKPGQSPKLLIYWASTRHTGVPDRFTGSGSGSGTDFTLTISNVQSENLADYFCQQYSTYPLTFGSGTKLEIKRADAAPTVSIFPPSSEQLTSGGASVVCFLNNFYPK

SEQ ID NO: 26 - protein, 196 aa, Mus musculus; identical to SEQ ID NO: 20

SEQ ID NO: 27 - protein, 11 aa, synthetic construct
  source 1..11: note = LCDR1-HL2434 Z-specific Abs (IgBLAST)
KASQDVGTAVA

SEQ ID NO: 28 - protein, 7 aa, synthetic construct
  source 1..7: note = LCDR2-HL2434 Z-specific Abs (IgBLAST)
WASTRHT

SEQ ID NO: 29 - protein, 9 aa, synthetic construct
  source 1..9: note = LCDR3-HL2434 Z-specific Abs (IgBLAST)
QQYSTYPLT

SEQ ID NO: 30 - protein, 5 aa, synthetic construct
  source 1..5: note = HCDR1-HL2434 Z-specific Abs (IgBLAST)
SYWIT

SEQ ID NO: 31 - protein, 17 aa, synthetic construct
  source 1..17: note = HCDR2-HL2434 Z-specific Abs (IgBLAST)
DIYPGSGSTNYNEKFKN

SEQ ID NO: 32 - protein, 7 aa, synthetic construct
  source 1..7: note = HCDR3-HL2434 Z-specific Abs (IgBLAST)
AVKGFAY

SEQ ID NO: 33 - protein, 11 aa, synthetic construct
  source 1..11: note = LCDR1-HL1314 M-specific Abs (IgBLAST)
KASQDINSYLS

SEQ ID NO: 34 - protein, 7 aa, synthetic construct
  source 1..7: note = LCDR2-HL1314 M-specific Abs (IgBLAST)
RANRLVD

SEQ ID NO: 35 - protein, 9 aa, synthetic construct
  source 1..9: note = LCDR3-HL1314 M-specific Abs (IgBLAST)
LQYDEFPYT

SEQ ID NO: 36 - protein, 5 aa, synthetic construct
  source 1..5: note = HCDR1-HL1314 M-specific Abs (IgBLAST)
SYWMN

SEQ ID NO: 37 - protein, 17 aa, synthetic construct
  source 1..17: note = HCDR2-HL1314 M-specific Abs (IgBLAST)
KIDPSDSETHYNQKFKD

SEQ ID NO: 38 - protein, 8 aa, synthetic construct
  source 1..8: note = HCDR3-HL1314 M-specific Abs (IgBLAST)
SPGGYPDW

(19) United States
(12) Patent Application Publication
(10) Pub. No.: US 2025/0208146 A1
(43) Pub. Date: Jun. 26, 2025

(54) ALPHA-1 ANTITRYPSIN Z- AND M-SPECIFIC BINDING PROTEINS

(71) Applicant: UNIVERSITY OF FLORIDA RESEARCH FOUNDATION, INCORPORATED, Gainesville, FL (US)

(72) Inventors: Mark Louis Brantly, Gainesville, FL (US); Alek Aranyos, Gainesville, FL (US); Tammy O. Flagg, Gainesville, FL (US)

(21) Appl. No.: 18/852,016

(22) PCT Filed: Mar. 27, 2023
(86) PCT No.: PCT/US23/64998

§ 371 Date: Sep. 27, 2024

Related U.S. Application Data
(60) Provisional application No. 63/324,548, filed on Mar. 28, 2022.

(51) Int. Cl. G01N 33/68; C07K 16/38; G01N 33/543

(52) U.S. Cl. CPC G01N 33/6893; C07K 16/38; G01N 33/54386; C07K 2317/33

(57) ABSTRACT

Binding proteins that bind Z-alpha-1 antitrypsin (Z-AAT) and M-alpha-1 antitrypsin (M-AAT) are provided, as well as kits comprising the binding protein(s). The disclosure also provides a method for detecting Z-alpha-1 antitrypsin (Z-AAT) in a subject, a method of characterizing delivery of M-AAT to a subject suffering from AAT deficiency, and a method of detecting AAT deficiency in a subject.

### FIELD

The present disclosure relates to binding proteins that bind alpha-1 antitrypsin (AAT) and methods of use.

### INCORPORATION BY REFERENCE OF MATERIALS SUBMITTED ELECTRONICALLY

This application contains, as a separate part of the disclosure, a Sequence Listing in computer readable form (Filename: 57311_SeqListing.txt; Size: 9,824 bytes; Created Mar. 25, 2023), which is incorporated by reference in its entirety.

### BACKGROUND

Alpha-1 antitrypsin (AAT) is a 52-kD glycoprotein encoded by the SERPINA1 gene, and is the most abundant circulating protease inhibitor in human plasma. The SERPNA1 gene is highly polymorphic; over 100 AAT variants have been described in various populations. AAT variants are classified according to the protease inhibitor (Pi) system of nomenclature, which is based on the banding pattern of AAT on polyacrylamide isoelectric focusing (IEF) gel electrophoresis. Variants are assigned a letter designation, corresponding to the rate at which the variant migrates toward the anode (i.e., “F” for fast, “M” for medium, “S” for slow, and “Z” for very slow). Some variants are also assigned a number or a name subscript (indicating the city in which the variant was first identified) when multiple subtypes are present in a given class.

A single amino acid substitution in the Z variant AAT molecule (Glu342Lys) causes abnormal folding of AAT, leading to polymerization and accumulation of Z-AAT within the endoplasmic reticulum leading to major clinical manifestations, including lung disease and liver disease. In cases of liver disease, patients with homozygous Z alleles have higher chances of developing neonatal hepatitis, fibrosis, cirrhosis, or hepatocellular carcinoma. In cases of lung disease, individuals homozygous for the AAT Z allele have a markedly increased risk of developing early on-set emphysema and/or abnormal liver function in infancy that may lead to complete liver failure which is linked to the lack of proteinase inhibitor and uncontrolled proteolysis.

The mean concentration of AAT in serum or plasma in healthy individuals is estimated to be 1.3-1.7 g/L, with a half-life of 3-5 days. Circulating AAT increases rapidly to concentrations exceeding 2 g/L in response to a wide range of inflammatory conditions, such as infections, cancer, liver disease, or pregnancy. When the AAT concentration in plasma decreases to<0.7 g/L, the individual is considered AAT-deficient. Alpha-1 antitrypsin deficiency (AATD), also known as alpha-1 proteinase inhibitor deficiency, is a hereditary autosomal disorder resulting from a variety of mutations in the alpha-1 antitrypsin (AAT) protein, leading to increased risk of lung and liver disease and several other conditions. The most common variant, PiM (i.e., AAT migrates in the middle), is present in 95% of the Caucasian U.S. population and is regarded as the variant associated with normal serum concentrations of functional AAT. The concentration of circulating AAT in the MM phenotype is therefore assigned a relative value of 100%. Heterozygous or homozygous combinations have AAT serum concentrations corresponding to 50% (MZ), 37.5% (SZ), 65% (SS), and 15% (ZZ) of this MM value, respectively. More than 90% of clinical cases of severe AAT deficiency are caused by the homozygous Z variant.

AATD can be diagnosed by genetic testing; however, AATD remains substantially under-diagnosed. It is estimated that approximately 90% of patients with AATD in the United States go undiagnosed due to several obstacles. AATD is often misdiagnosed as chronic obstructive pulmonary disease (COPD) and cryptogenic liver disease due to similar clinical manifestations. Other reasons for underdiagnoses include a lack of awareness of the condition, a lack of availability of a nationwide AATD screening program, a lack of knowledge about the tests necessary for the diagnosis, and the lack of availability of such tests. Improving the detection rate for AATD is a high priority to reduce the time to diagnosis, which improves outcomes, and also to increase the pool of those with confirmed diagnoses for recruitment to clinical trials.

Currently, AATD testing is based on the combination of different laboratory methods, such as measuring the AAT concentration in serum, followed by a phenotyping and genotyping analysis. However, measurement of serum AAT by quantitative immunoprecipitation is insufficient for the diagnosis of AATD because the concentration of the protein is known to increase during the acute-phase response, pregnancy, cancer, or other conditions, and thus can mask a partial AAT deficiency. Phenotypic analysis performed by IEF or agarose gel electrophoresis with immunofixation are fairly cumbersome to perform, and interpretation of the gel pattern requires special training and skills. On the other hand, genotyping methods are not routinely available in diagnostic laboratories because they are expensive, require special skills, and are not well suited for screening purposes. Therefore, there is need for the development of new, time and cost effective methodologies allowing efficient and reliable testing, including point-of-care testing (POCT), of AATD.

### SUMMARY

The present disclosure provides binding proteins, e.g., antibodies or antigen binding fragments thereof, that specifically bind to human Alpha-1 Antitrypsin (AAT) proteins. In one aspect, the disclosure provides a binding protein (e.g., an antibody or antigen binding fragment thereof) that specifically binds to Z Alpha-1 Antitrypsin (Z-AAT). In some embodiments, the binding protein comprises a set of six CDRs set forth in SEQ ID NOs: 11-16. A method for detecting Z-alpha-1 antitrypsin (Z-AAT) in a subject also is contemplated. The method comprises contacting a biological sample from a subject with the disclosed binding protein that binds Z-AAT, and detecting binding of the binding protein to Z-AAT in the sample.

In another aspect, the disclosure provides a binding protein (e.g., an antibody or antigen binding fragment thereof) that specifically binds M Alpha-1 Antitrypsin (M-AAT). In some embodiments, the binding protein (e.g., antibody or antigen binding fragment thereof) comprises a set of six CDRs set forth in SEQ ID NOs: 3-8. The disclosure further provides a method of characterizing delivery of M-AAT to a subject suffering from AAT deficiency. The method comprises contacting a biological sample from a subject with the binding protein that binds M-AAT. The subject is homozygous for Z-AAT and previously administered aerosolized M-AAT. The method further comprises detecting binding of the binding protein to M-AAT in the sample, wherein binding indicates that the aerosolized M-AAT has reached the alveolar space of the subject.

The disclosure further provides solid supports comprising the binding proteins described herein, as well as kits comprising any of the components described herein. For example, the disclosure provides a kit comprising a solid support wherein the Z-AAT binding protein described herein is immobilized at a first position and the M-AAT binding protein described herein is immobilized at a second position. For instance, the disclosure provides a solid support comprising a sample application zone; a detection antibody zone adjacent to the sample application zone, wherein detection antibodies are adhered to the solid support such that the detection antibodies are released upon contact with a biological sample and/or buffer; a test zone adjacent to the detection antibody zone, wherein the test zone comprises the Z-AAT binding protein immobilized in a first position and the M-AAT binding protein immobilized in a second position; a control zone adjacent to the test zone, wherein the control zone comprises capture antibodies which bind the detection antibody immobilized on the solid support, and a wick. A method for detecting AATD in a subject using the disclosed solid support also is contemplated. The method comprises adding a sample obtained from a subject to the solid support wherein, when the assay system exhibits a single band at the first position of the test zone, the subject is determined as being homozygous for Z-AAT, when the assay system exhibits a band in each of the first position and the second position, the subject is determined as being heterozygous for Z-AAT and M-AAT, and when the assay system exhibits a band in the second position of the test zone and a band is absent in the first position, the subject is determined as being homozygous for M-AAT.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. Section headings, if present, are provided merely for the convenience of the reader. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF THE DRAWINGS

FIG. 1 is a Western blot demonstrating specific binding of antibody M1-2012 to M-AAT protein with little to no cross reactivity with Z-AAT protein, where purified Z-AAT and M-AAT proteins were probed with purified M1-2012.

FIG. 2 is a Western blot demonstrating the specific binding of Z-specific antibodies HL2440, HL2434, HL2442, HL2438, and HL2444 to Z-AAT protein with little to no cross reactivity with M-AAT protein, where a purified Z-specific antibody was used to probe purified Z-AAT and M-AAT proteins.

FIG. 3 is a dot blot analysis showing detection of Z-AAT purified protein by hybridoma supernatant containing Z-specific monoclonal antibodies which demonstrated little to no cross-reactivity with purified M-AAT protein. Blot further shows specific detection of purified M-AAT protein by the purified M-specific antibody, while the “Total-AAT” antibody detects both Z and M-AAT proteins.

FIG. 4 is a native Western blot analysis showing specific detection of Z-AAT and M-AAT in plasma samples using purified Z-specific and M-specific antibodies. The Z-specific antibody showed only minor cross-reactivity with M-AAT plasma samples while the M-specific antibody showed weak cross-reactivity with plasma Z-AAT.

FIG. 5 is a graph providing ELISA curves illustrating the ability of the Z-specific antibody HL2440 to specifically bind Z-AAT protein in plasma with little to no cross reactivity with plasma containing the M-AAT protein.

FIG. 6 is a graph providing ELISA curves illustrating the ability of the Z-specific antibody HL2440 to specifically bind AAT variant proteins in plasma from ZZ homozygotes as well as plasma from MZ and SZ heterozygotes, with little to no cross reactivity with MM plasma.

FIG. 7 is a graph comparing standard curves correlating Z-AAT antigen concentration in plasma with the detection signal from an ELISA assay with the Z-specific antibody. The graph shows an increase in the dynamic range with the use of HEPES buffer prior to and during the association step. The dynamic range also increased with HEPES buffer pH 7.4 versus pH 7.9.

FIG. 8 is a bar graph illustrating the cross reactivity of a Z-specific antibody of the disclosure in binding non-ZAAT protein in S/S, Ma/Ma or M/M homozygote plasma under different conditions using ELISA and presented as percent of nephelometric value. The Cross Reactivity Index represents the (ELISA values interpolated from the Z-standard curve/Nephelometer derived values for each condition)×100. A lower percent of non-Z-AAT bound is indicative of better specificity of the Z-specific antibody. Parameters varied in the assay include HEPES Buffers, pH, and wash buffer combinations. This provides a measure of specificity of Z-specific antibody for other AAT serotypes when compared with the Z-AAT derived standard curve. All plasma samples were tested in duplicate at a 1/40 dilution. Under the conditions tested, the greatest level of antibody specificity was observed at pH 7.4.

FIG. 9 provides binding sensorgrams from BLI-Octet binding affinity analysis between Z-AAT protein variant and Z-specific antibodies from different hybridoma clones in HEPES buffer. All the antibodies except HL2444 had equilibrium dissociation constants (Kd) in the range of 10⁻⁷ to 10⁻⁹.

FIG. 10 provides alignments of light chain variable region sequences of Z-specific antibodies and an M-specific antibody of the disclosure. CDRs are predicted utilizing CDRs from a sequence (GenBank: AB022779.1) with high consensus the M-specific antibody (HL1314), and by utilizing the Paratome Analysis Software. Predicted CDRs are highlighted. It will be appreciated that alternative software programs may identify slightly different boundaries for CDR regions. CDR regions identified using IgBLAST of Z-specific antibodies are provided as SEQ ID NOs: 27-32. CDR regions identified using IgBLAST of M-specific antibodies are provided as SEQ ID NOs: 33-38.

FIG. 11 provides alignments of heavy chain variable region sequences of Z-specific antibodies and an M-specific antibody of the disclosure. Predicted CDRs are highlighted. CDR1* is predicted utilizing CDRs from a sequence (GenBank: AJ012555.1) with high consensus with the Z-specific antibody (HL2434), and by utilizing the Abysis Software and comparing the outputs from multiple region definitions.

FIG. 12 is a schematic illustration of a solid support for a representative lateral flow assay for detecting the presence of M-AAT or Z-AAT protein in a sample.

FIG. 13 is a table listing various amino acid sequences referenced herein.

### DETAILED DESCRIPTION

The disclosure provides binding proteins that bind Z-alpha-1 antitrypsin (Z-AAT) and binding proteins that bind M-alpha-1 antitrypsin (M-AAT). The binding proteins of the disclosure may be used in a variety of contexts, such as in methods of detecting or quantifying M-AAT or Z-AAT, detecting AAT deficiency, characterizing patients that display symptoms of AAT deficiency, determining the effectiveness of delivery of M-AAT to subjects, and the like. The disclosure provides assay systems and kits, which are useful for these and other applications (e.g., for detecting Z-AAT or M-AAT in a sample, monitor M-AAT levels in AATD patients undergoing aerosolized, AAT replacement therapy, and the like).

### Binding Proteins

Z-AAT and M-AAT are well characterized in the art. In exemplary aspects, the binding proteins bind human Z-AAT or human M-AAT. A representative amino acid sequence of Z-AAT is set forth in SEQ ID NO: 1. A representative amino acid sequence of M-AAT is set forth in SEQ ID NO: 2. In various aspects, the binding protein “specifically binds” Z-AAT or M-AAT. “Specifically binds” and “specific binding” means that the binding protein (e.g., antibody or antigen binding fragment) preferentially binds Z-AAT or M-AAT over other proteins. In this regard, an agent “specifically binds” to a target if it reacts or associates more frequently, more rapidly, with greater duration and/or with greater affinity with a particular target (e.g., Z-AAT) than it does with alternative targets (e.g., M-AAT). For example, the Z-AAT binding protein is optionally one that binds Z-AAT with greater affinity, avidity, more readily, and/or with greater duration than it binds to other non-Z-AAT proteins, such as M-AAT. Similarly, the M-AAT binding protein is optionally one that binds M-AAT with greater affinity, avidity, more readily, and/or with greater duration than it binds to other non-M-AAT proteins, such as Z-AAT. In various aspects of the disclosure, the binding protein that specifically binds M-AAT demonstrates minimal or no cross reactivity with Z-AAT, and binding protein that binds Z-AAT demonstrates minimal or no cross reactivity with M-AAT.

For example, in some various aspects, a binding protein that “specifically binds” Z-AAT or M-AAT demonstrates a higher affinity for Z-AAT or M-AAT compared to other proteins. Binding proteins (e.g., antibodies or fragments thereof) that bind an antigen, such as Z-AAT or M-AAT may have a binding affinity for the antigen of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. It will be appreciated that ranges having the values above as end points is contemplated in the context of the disclosure. For example, the binding protein (e.g., antibody or antigen binding fragment thereof) may bind Z-AAT of SEQ ID NO: 1 or M-AAT of SEQ ID NO: 2 with an affinity of about 1×10⁻⁷ M to about 1×10⁻¹² M or an affinity of about 1×10⁻⁹ to about 1×10⁻¹² or an affinity of about 1×10⁻⁷ M to about 1×10⁻⁹ M. In various aspects, the binding affinity of the Z-AAT binding protein to Z-AAT is at least about 2×, 5×, 10×, 20×, 30×, 50×, 75×, or 100× the affinity of the binding protein to M-AAT. Conversely, in various aspects, the binding affinity of the M-AAT binding protein to M-AAT is at least about 2×, 5×, 10×, 20×, 30×, 50×, 75×, or 100× the affinity of the binding protein to Z-AAT.

Methods of characterizing the binding of binding proteins, such as antibodies, to a target are known in the art and include, e.g., radioimmunoassay (RIA), ELISA, Western blot, immunoprecipitation, surface plasmon resonance (e.g., BIAcore), Biolayer Interferometry (BLI), and competitive inhibition assays (see, e.g., Janeway et al., infra; U.S. Patent Application Publication No. 2002/0197266; and U.S. Pat. No. 7,592,429). In exemplary aspects, the binding proteins provided herein exhibit sufficient affinity for Z-AAT or M-AAT in a human biological sample, such as blood, serum, or plasma, so as to detect the target protein in a biological sample. In exemplary aspects, the Z-AAT binding protein binds to Z-AAT and does not detectably bind to M-AAT in a sample. In exemplary aspects, the M-AAT binding protein binds to M-AAT and does not detectably bind to a Z-AAT in a sample.

The term “binding protein” includes antibodies, antigen-binding fragments of antibodies, and antibody-like protein constructs. The term “antibody” refers to an intact antigen-binding immunoglobulin. In various embodiments, an intact antibody comprises two full-length heavy chains and two full-length light chains. In a full-length antibody, each heavy chain is comprised of a heavy chain variable region (abbreviated herein VH) and a heavy chain constant region. The heavy chain constant region is comprised of three domains, CH1, CH2 and CH3. Each light chain is comprised of a light chain variable region (abbreviated herein VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG 3, IgG4, IgA1 and IgA2) or subclass.

The binding protein may, in various aspects, be an “antigen-binding fragment” of an antibody, i.e., a fragment of an antibody that retains the ability to bind to Z-AAT or M-AAT. Examples of antigen-binding fragments of antibodies include, but are not limited to (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) an Fv fragment consisting of the VL and VH domains of a single arm of an antibody, (v) a dAb fragment (Ward et al., (1989) Nature 341:544-546, Winter et al., PCT Publication No. WO 90/05144), which comprises a single variable domain. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies (scFv) are also intended to be encompassed within the term “antigen-binding fragment” of an antibody.

The architecture of antibodies has been exploited to create a growing range of alternative formats that span a molecular-weight range of at least about 12-150 kDa and have a valency (n) range from monomeric, to dimeric, to trimeric, to tetrameric, and potentially higher; such alternative formats are referred to herein as “antibody-like constructs.” Antibody-like protein constructs include those based on the full antibody structure and those that mimic antibody fragments which retain full antigen-binding capacity, e.g., scFvs, Fabs, and VHH. The smallest antigen-binding fragment that retains its complete antigen binding site is the Fv fragment, which consists entirely of variable (V) regions. Other antibody-like protein constructs include disulfide-bond stabilized scFv (ds-scFv), single chain Fab (scFab), as well as di-and multimeric antibody formats like dia-, tria-and tetra-bodies, or minibodies (miniAbs) that comprise different formats consisting of scFvs linked to oligomerization domains. The smallest fragments are VHH/VH of camelid heavy chain Abs as well as single domain Abs (sdAb). A building block that is frequently used to create different antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody-like construct protein product. The structure of a peptibody consists of a biologically active peptide grafted onto an Fc domain. Peptibodies are described in the art. See, e.g., Shimamoto et al., mAbs 4(5): 586-591 (2012). Other antibody-like protein constructs include a single chain antibody (SCA), a diabody, a triabody, a tetrabody, and the like.

The binding protein may be a multi-specific antibody (e.g., a bispecific antibody or trispecific antibody) having the CDR sequences set forth herein. Bispecific antibody products can be divided into five major classes: BsIgG, appended IgG, BsAb fragments (e.g., bispecific single chain antibodies), bispecific fusion proteins (e.g., antigen binding domains fused to an effector moiety), and BsAb conjugates. See, e.g., Spiess et al., Molecular Immunology 67(2) Part A: 97-106 (2015). Examples of bispecific antibody constructs include, but are not limited to, tandem scFvs and Fab2 bispecifics. See, e.g., Chames & Baty, 2009, mAbs 1[6]:1-9; and Holliger & Hudson, 2005, Nature Biotechnology 23[9]:1126-1136; Wu et al., 2007, Nature Biotechnology 25[11]:1290-1297; Michaelson et al., 2009, mAbs 1[2]:128-141; International Patent Publication No. WO 2009032782 and WO 2006020258; Zuo et al., 2000, Protein Engineering 13[5]:361-367; U.S. Patent Application Publication No. 20020103345; Shen et al., 2006, J Biol Chem 281[16]:10706-10714; Lu et al., 2005, J Biol Chem 280[20]:19665-19672; and Kontermann, 2012 MAbs 4(2):182, all of which are expressly incorporated herein. Multispecific antibody constructs, such as trispecific antibody constructs (including three binding domains) or constructs having more than three (e.g., four, five, or more) specificities also are contemplated.

The antibodies (or antigen-binding fragments thereof or antibody-like protein constructs) may be a human antibody (i.e., having one or more variable and constant regions derived from human immunoglobulin sequences), humanized (i.e., have a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject), or chimeric (i.e., containing one or more regions from one antibody and one or more regions from one or more other antibodies).

In various aspects, the binding protein that binds to human Z Alpha-1 Antitrypsin (Z-AAT) comprises at least one CDR sequence having at least 75% identity (e.g., at least 75%, 80%, 85%, 90%, 95% or 100% identity) to a CDR selected from HCDR1, HCDR2, HCDR3, LCDR1, LCDR2, and LCDR3 wherein HCDR1 has the sequence given in SEQ ID NO: 14, HCDR2 has the sequence given in SEQ ID NO: 15, HCDR3 has the sequence given in SEQ ID NO: 16, LCDR1 has the sequence given in SEQ ID NO: 11, LCDR2 has the sequence given in SEQ ID NO: 12, and LCDR3 has the sequence given in SEQ ID NO: 13. The Z-AAT binding protein, in various aspects, comprises two of the CDRs, three of the CDRs, four of the CDRs, five of the CDRs or all six of the CDRs. Alternatively or in addition, the Z-AAT binding protein comprises CDRs having the amino acid sequences set forth in SEQ ID NOs: 11-16 wherein one, two, or three of the amino acids in any one or more of the CDR sequences are substituted. In an exemplary embodiment, the Z-AAT binding protein comprises a set of six CDRs as follows: HCDR1 of SEQ ID NO: 14, HCDR2 of SEQ ID NO: 15, HCDR3 of SEQ ID NO: 16, LCDR1 of SEQ ID NO: 11, LCDR2 of SEQ ID NO: 12, and LCDR3 of SEQ ID NO: 13.

In some or any aspects, the binding protein comprises a light chain variable region comprising an amino acid sequence having at least 75% identity (e.g., at least 75%, 80%, 85%, 90%, 95% or 100% identity) to the amino acid sequence set forth in SEQ ID NO: 17, 19, 21, 23, or 25 and/or a heavy chain variable region comprising an amino acid sequence having at least 75% identity (e.g., at least 75%, 80%, 85%, 90%, 95% or 100% identity) to the amino acid sequence set forth in SEQ ID NO: 18, 20, 22, 24, or 26 (e.g., comprising the amino acid sequences of SEQ ID NOs: 17 and 18, comprising the amino acid sequences of SEQ ID NOs: 19 and 20, comprising the amino acid sequences of SEQ ID NOs: 21 and 22, comprising the amino acid sequences of SEQ ID NOs: 23 and 24, or comprising the amino acid sequences of SEQ ID NOs: 25 and 26). In various aspects, the difference in the sequence compared to SEQ ID NO: 17, 19, 21, 23, or 25 or SEQ ID NO: 18, 20, 22, 24, or 26 lies outside the CDR region in the corresponding sequences. In some or any embodiments, the binding protein (e.g., antibody or antigen binding fragment) comprises a light chain variable region comprising an amino acid sequence set forth in SEQ ID NO: 17 and a heavy chain variable region comprising an amino acid sequence set forth in SEQ ID NO: 18. The disclosure further contemplates a binding protein (e.g., an antibody or fragment thereof) comprising the CDR sequences within the variable light chain sequences of SEQ ID NOs: 17, 19, 21, 23, or 25 and/or variable heavy chain sequences of SEQ ID NO: 18, 20, 22, 24, or 26.

In various aspects, the binding protein that binds to human M Alpha-1 Antitrypsin (M-AAT) comprises at least one CDR sequence having at least 75% identity (e.g., at least 75%, 80%, 85%, 90%, 95% or 100% identity) to a CDR selected from HCDR1, HCDR2, HCDR3, LCDR1, LCDR2, and LCDR3 wherein HCDR1 has the sequence given in SEQ ID NO: 6, HCDR2 has the sequence given in SEQ ID NO: 7, HCDR3 has the sequence given in SEQ ID NO: 8, LCDR1 has the sequence given in SEQ ID NO: 3, LCDR2 has the sequence given in SEQ ID NO: 4, and LCDR3 has the sequence given in SEQ ID NO: 5. The M-AAT binding protein, in various aspects, comprises two of the CDRs, three of the CDRs, four of the CDRs, five of the CDRs or all six of the CDRs. Alternatively or in addition, the M-AAT binding protein comprises CDRs having the amino acid sequences set forth in SEQ ID NOs: 3-8 wherein one, two, or three of the amino acids in any one or more of the CDR sequences are substituted. In an exemplary embodiment, the M-AAT binding protein comprises a set of six CDRs as follows: HCDR1 of SEQ ID NO: 6, HCDR2 of SEQ ID NO: 7, HCDR3 of SEQ ID NO: 8, LCDR1 of SEQ ID NO: 3, LCDR2 of SEQ ID NO: 4, and LCDR3 of SEQ ID NO: 5.

In some or any embodiments, the M-AAT binding protein comprises a light chain variable region comprising an amino acid sequence having at least 75% identity (e.g., at least 75%, 80%, 85%, 90%, 95% or 100% identity) to the amino acid sequence set forth in SEQ ID NO: 9 and/or a heavy chain variable region comprising an amino acid sequence having at least 75% identity (e.g., at least 75%, 80%, 85%, 90%, 95% or 100% identity) to the amino acid sequence set forth in SEQ ID NO: 10 (e.g., comprising the amino acid sequence of SEQ ID NO: 9 and 10). In various aspects, the difference in the sequence compared to SEQ ID NO: 9 or 10 lies outside the CDR region in the corresponding sequences. In some or any embodiments, the M-AAT binding protein (e.g., antibody or antigen binding fragment) comprises a light chain variable region comprising an amino acid sequence set forth in SEQ ID NO: 9 and a heavy chain variable region comprising an amino acid sequence set forth in SEQ ID NO: 10. The disclosure further contemplates a binding protein (e.g., an antibody or fragment thereof) comprising the CDR sequences within the variable heavy and/or variable light chain sequences of SEQ ID NOs: 9 or 10.

The disclosure also provides a binding protein (e.g., an antibody, antigen-binding antibody fragment, or antibody-like protein construct) which competes with the binding proteins described herein for binding to Z-AAT or M-AAT. Such binding proteins are often referenced as “cross-blocking binding proteins.” Cross-blocking assays are described in, e.g., U.S. Pat. No. 7,592,429, incorporated herein by reference.

Suitable methods of making antibodies are known in the art. For instance, standard hybridoma methods are described in, e.g., Harlow and Lane (eds.), Antibodies: A Laboratory Manual, CSH Press (1988), and CA. Janeway et al. (eds.), Immunobiology, 5th Ed., Garland Publishing, New York, NY (2001)). Monoclonal antibodies for use in the methods of the disclosure may be prepared using any technique which provides for the production of antibody molecules by continuous cell lines in culture. These include but are not limited to the hybridoma technique originally described by Koehler and Milstein (Nature 256: 495-497, 1975), the human B-cell hybridoma technique (Kosbor et al., Immunol Today 4:72, 1983; Cote et al., Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806,5,569,825, and 5,714,352, and U.S. Patent Application Publication No. 2002/0197266 AI). Antibodies may also be produced by inducing in vivo production in the lymphocyte population or by screening recombinant immunoglobulin libraries or panels of highly specific binding reagents as disclosed in Orlandi et al (Proc Natl Acad Sci 86:3833-3837; 1989), and Winter G and Milstein C (Nature 349:293-299, 1991). If the full sequence of the antibody or antigen-binding fragment is known, then methods of producing recombinant proteins may be employed. See, e.g., “Protein production and purification” Nat Methods 5(2): 135-146 (2008). In some embodiments, the antibodies (or antigen binding fragments) are isolated from cell culture or a biological sample if generated in vivo.

In various aspects, the binding protein of the disclosure is conjugated or attached to a reporter moiety, i.e., a moiety that provides a detectable signal. The reporter moiety can be any of a wide range of moieties/components of reporter or detection systems, such as those known in the art. In some aspects, the reporter moiety may comprise, e.g., an enzyme (e.g., horseradish peroxidase (HRP), alkaline phosphatase, luciferase, β-galactosidase, glucose oxidase, lysozyme, malate dehydrogenase, glucose-6-phosphate dehydrogenase); metal sol, selenium sol, carbon sol, and the like; colored or colorable particles (e.g., colored or colorable latex particles); radioactive moieties; or colloidal metal particles (e.g., colloidal gold, colloidal silver, colloidal platinum, colloidal selenium). Examples of methods for detecting reporter moieties include, but are not limited to, visible inspection, ultraviolet (UV) and visible spectrophotometry, fluorimetry, and radiation counters. The reporter moiety may be covalently or non-covalently bound/coupled to the binding protein. The binding/coupling can be accomplished by any method known in the art. For example, reagents used for binding/coupling include, but are not limited to, glutaraldehyde, p-toluene diisocyanate, various carbodiimide reagents, p-benzoquinone m-periodate, N,Ni-o-phenylenedimaleimide, recombinant methods, and the like.

### Solid Supports and Kits

The disclosure provides a solid support comprising the Z-AAT binding protein, the M-AAT binding protein, or a combination of Z-AAT and M-AAT binding proteins. Any solid support suitable for biological applications is appropriate for use in the context of the disclosure. For example, solid supports include, but are not limited to, a tube, a dish, a flask, a bag, a plate (e.g., a micro-well or microtiter plate), a test strip, a membrane, a filter, a bead (including micro-or nano-particle), a dipstick, a fiber, and the like. In exemplary aspects, the solid support is made of a polymer. In exemplary aspects, the solid support comprises agarose, cellulose, dextran, polyacrylamide, latex, or controlled pore glass. In exemplary aspects, the solid support is composed of polyvinyl difluoride (PVDF), nitrocellulose, nylon 66, protran nitrocellulose, or paper. In exemplary aspects, the solid support comprises a membrane, which is selected from Immobilon®, Protran®, QuickDraw®, Westran®, Whatman® or Hybond® membranes (Sigma-Aldrich, St. Louis, MO). Optionally, the solid support comprises pre-aliquoted amounts of the binding protein. The binding protein may be adhered to the solid support using any suitable method so long as the binding protein retains ability to bind the target (Z-AAT or M-AAT). The binding protein may be applied in a manner which allows release of the binding protein upon exposure to a reagent (or other environmental condition). Alternatively, the binding protein may be applied to the solid support in a manner which allows the binding protein to remain adhered to the support after exposure to a reagent (or other environmental condition).

The solid support is useful in a variety of contexts, including in methods of detecting or quantifying Z-AAT or M-AAT in a sample. If desired, the solid support may be configured in a lateral flow assay format and/or in an immunochromatographic assay format. Lateral flow assay systems are known in the art. See, e.g., Grant et al., Vaccine 34(46): 5656-5663 (2016); and Cross et al., J Infect Dis 214(suppl3):S210-S217 (2016).

In various aspects, the solid support comprises the Z-AAT binding protein (i.e., the Z-AAT binding protein is immobilized on the solid support) and an M-AAT binding protein. Optionally, the Z-AAT binding protein and the M-AAT binding protein are antibodies, such as antibodies having the CDR sequences described herein. In instances where both Z-AAT and M-AAT binding proteins are present, the two types of binding proteins may be interspersed (i.e., applied in an overlapping manner) on the solid support or may be present in discrete locations. In this regard, in various aspects of the disclosure, the Z-AAT binding protein is immobilized at a first position on the surface of a solid support and an M-AAT binding protein (e.g., the M-AAT binding protein described herein) is immobilized at a second position on the surface of the solid support. The first and second positions are optionally located on the solid support to allow distinct detection of binding to Z-AAT to the Z-AAT binding protein and/or detection of M-AAT to the M-AAT binding protein. In various aspects of the disclosure, the solid support further comprises an anti-AAT detection antibody that binds both M-AAT and Z-AAT, and which is bound to a detection agent. The anti-AAT detection antibody is not the Z-AAT or M-AAT binding protein of the disclosure; it is a distinct binding protein which, in exemplary aspects, binds both Z-AAT and M-AAT. The anti-AAT detection antibody does not interfere with the Z-AAT binding protein or M-AAT binding protein's ability to bind Z-AAT or M-AAT (i.e., the anti-AAT detection antibody binds AAT at a different epitope). Optionally, the anti-AAT detection antibody is immobilized on the solid support in a fashion which allows the anti-AAT detection antibody to be released when exposed to, e.g., a fluid, such as a biological sample or liquid reagent (such as buffer). The detection agent may be any suitable agent that provides a detectable signal, such as visible (e.g., color) signal or radioactive signal. The anti-AAT detection antibody provides confirmation that AAT is present in the sample. In various aspects of the disclosure, the solid support further comprises a capture antibody which binds the anti-AAT detection antibody.

An exemplary solid support is arranged as follows. For illustration, the exemplary solid support is described as a test strip with a proximal and distal end, but it will be appreciated that other shapes, sizes, arrangements, and formats are also appropriate. For purposes of illustration, the test strip comprises a porous matrix (e.g., a membrane) which allows movement of a fluid sample along the length of the test strip, the matrix optionally adhered to a more rigid backing to facilitate handling. In the exemplary embodiment, the solid support comprises a sample application zone. The sample application zone is a region of the test strip upon which a sample (e.g., a biological sample or other type of sample in which Z-AAT and/or M-AAT may be present) is applied. Adjacent to the sample application zone is a detection antibody zone. The detection antibody zone contains immobilized anti-AAT antibodies, which bind both Z-AAT and M-AAT. The detection antibodies are adhered to the solid support such that the detection antibodies are released upon contact with a biological sample and/or buffer. The solid support further comprises a test zone adjacent to the detection antibody zone. The test zone comprises the Z-AAT binding protein described herein in a first position and the M-AAT binding protein described herein in a second position. The binding proteins are immobilized on the solid support in the test zone in such a matter that the binding proteins bind their target (Z-AAT or M-AAT) when exposed to the sample, thereby creating a zone with antibody-target complexes when the target is present in the sample. The solid support further comprises a control zone adjacent to the test zone. The control zone comprises capture antibodies which bind the detection antibody, thereby providing confirmation that the detection antibodies were released from the solid support. Finally, the solid support optionally comprises a wick, which facilitates movement of the sample (or components thereof) into or along the test strip.

Merely to illustrate the operation of this exemplary embodiment, a sample is applied to the sample application zone, which is optionally impregnated with buffer or surfactant or other reagent which promotes maintenance of target protein conformation or distribution of the sample along the solid support. The sample migrates along the test strip by capillary action to the detection antibody zone, thereby releasing the anti-AAT detection antibodies. The sample migrates to the test zone which comprises the Z-AAT binding protein and M-AAT binding protein immobilized in two test lines (or spots or other shape suitable for detection). Z-AAT present in the sample is captured by the Z-AAT antibody, and M-AAT present in the sample is captured by the M-AAT binding protein. Binding of the AAT target protein to its binding protein (the complex) is identified by detecting binding of the anti-AAT detection antibody to the complex. For example, a color reaction is indicated in the test zone by the binding protein+AAT+anti-AAT detection antibody complex. The control zone provides a means for confirming that the assay is functioning correctly insofar as released anti-AAT antibodies are bound by the capture antibodies, which can be visualized, thereby confirming that the anti-AAT detection antibodies were available for detecting binding protein-AAT complexes in the test zone. In this illustrative embodiment, the read-out is represented by color regions (i.e., lines) appearing at different locations on the test strip (optionally with different intensities depending on AAT concentration) to signal that Z-AAT and/or M-AAT is present. The color regions can be assessed by eye or using a dedicated reader. The absence of a color in the control region indicates an invalid test result.

Also provided herein are kits comprising any one or more of the binding proteins described herein, optionally adhered to a solid support. Kits generally comprise one or more binding proteins of the disclosure in or on a suitable container or support alongside instructions for use, and optionally further comprising reagents for use in connection with the binding proteins. Optionally, the binding protein(s) is provided in a predetermined amount or concentration. The binding protein(s) may be provided in the kit in the form of an aqueous solution, a frozen composition, or a lyophilized or other freeze-dried form.

In exemplary aspects, the kit comprises additional reagents, such as substrates, solvents, buffers, diluents, etc., for a particular use of the binding protein. Examples of additional reagents include, but are not limited to, a blocking agent, such as, for example, a solution comprising bovine serum albumin (BSA); buffer, such as, for example, phosphate buffered saline or TRIS buffer; and/or a detecting agent. Suitable detecting agents are known in the art and described herein. Detecting agents include, but are not limited to, a secondary antibody linked to a detectable label (e.g., an enzyme, such as horseradish peroxidase (HRP)). The secondary antibody optionally binds the Z-AAT and/or M-AAT binding protein. In instances where an enzyme is employed in the kit for detection purposes, the kit may further comprise the enzyme's substrate (e.g., a chromogenic substrate). Examples of enzyme substrates are known in the art and include, e.g., 2,2′-azino-bis(3-ethylbenzothiazoline-6-sulphonic acid) (ABTS), o-phenylenediamine dihydrochloride (OPD), AmplexRed, 3,3′-Diaminobenzidine (DAB), aminoethyl carbazole (AEC), 3,3′,5,5′-Tetramethylbenzidine (TMB), Homovanillic acid, and Luminol. In exemplary aspects, the kit comprises reagents and materials for an ELISA, e.g., a sandwich ELISA, or other type of immunoassay. For example, the kit may comprise a Z-AAT-specific antibody adhered to a solid support (e.g., a microtiter plate or nitrocellulose) coated with a capture molecule, a blocking agent (e.g., BSA), and a detecting agent.

### Methods of Use

The binding protein provided herein is useful in, e.g., methods for detecting Z-AAT and/or M-AAT in samples, such as biological samples. In one aspect, the disclosure provides a method for detecting Z-alpha-1 antitrypsin (Z-AAT) in a subject, the method comprising contacting a biological sample from a subject with the Z-AAT binding protein described herein and detecting binding of the binding protein to Z-AAT in the sample. The present disclosure also provides a method of detecting M-AAT in a sample obtained from a subject. In exemplary embodiments, the method comprises contacting the sample with the M-AAT binding protein described herein to form a complex (e.g., an immunocomplex) and detecting the complex. When the complex is detected, it is determined that the sample comprises M-AAT.

Additionally, the disclosure provides a method of characterizing delivery of M-AAT to a subject suffering from AAT deficiency. The method comprises contacting a biological sample with the M-AAT binding protein described herein. The biological sample is from a subject homozygous for Z-AAT and previously administered aerosolized M-AAT. Subjects homozygous for Z-AAT do not naturally produce M-ATT. The method further comprises detecting binding of the binding protein to M-AAT in the sample. The presence of binding (e.g., the presence of M-AAT complexed to M-AAT binding protein) indicates that the aerosolized M-AAT has reached the alveolar space of the subject. Thus, the disclosure provides materials and methods well suited for characterizing the delivery of M-AAT therapeutic to a subject suffering from AATD.

Detection methodologies utilizing binding proteins to identify a target protein in a sample are known in the art and include, e.g., radioimmunoassay (RIA), magnetic immunoassay (MIA), immunocytochemical (ICC) assays, immunohistochemical (IHC) assays, immunofluorescent assays, ELISA, EIA, ELISPOT, enzyme multiplied immunoassay, radiobinding assay, Western blotting, immunoprecipitation, dot blots, flow cytometry, real-time immunoquantitative PCR, protein microarrays, and the like. See, e.g., The Immunoassay Handbook (Fourth Edition); Theory and Applications of Ligand Binding, ELISA and Related Techniques, ed. Wild, Elsevier Ltd. (Oxford, UK) 2013, Green and Sambrook, Molecular Cloning: A Laboratory Manual, 4th ed., Cold Spring Harbor Laboratory Press (Cold Spring Harbor, NY) 2012, and Immunoassay, Diamandis and Christopolous, Academic Press 1996.

A biological sample is, in various aspects, obtained from a human (or other mammalian subject), for example, by collecting a bodily fluid sample or swabbing a body orifice. The sample may be collected by, e.g., a health care work or self-sampling. The biological sample may be blood, interstitial fluid, plasma, serum, urine, cerebral spinal fluid, sweat, saliva, or other clinically relevant sample, which may be processed (if desired) to remove various components naturally found in the sample other than AAT. Pre-treatment of the sample can involve filtration, precipitation, dilution, distillation, concentration, inactivation of interfering components, and the addition of reagents. A solid material suspected of containing AAT can be used as the source of the sample, preferably by modifying the solid material to form a liquid or semi-liquid composition. In some embodiments, the sample is an undiluted sample, i.e., the sample is obtained from the biological source and directly tested without any pre-dilution of the sample.

In another aspect, the disclosure provides a method of detecting AAT deficiency in a subject, comprising adding a sample obtained from a subject to the solid support described herein. For example, the solid support comprises a sample application zone; a detection antibody zone, wherein anti-AAT detection antibodies are adhered to the solid support such that the detection antibodies are released upon contact with a biological sample and/or buffer; a test zone, wherein the test zone comprises the Z-AAT binding protein described herein in a first position and the M-AAT binding protein described herein in a second position; and a control zone, wherein the control zone comprises capture antibodies immobilized on the solid support and which bind the anti-AAT detection antibody. In various aspects, the method further comprises detecting an observable signal on the solid surface indicative of (i) the presence of Z-AAT+Z-AAT binding protein+anti-AAT detection antibody complexes, (ii) the presence of M-AAT+M-AAT binding protein+anti-AAT detection antibody complexes, and/or (iii) capture antibody+anti-AAT detection antibody complexes. When the solid support exhibits a detectable signal in the first position of the test zone and not the second position, the subject is determined as being homozygous for Z-AAT. Homozygosity for Z-AAT is indicative of AAT deficiency. When the solid support exhibits a detectable signal in each of the first position and the second position, the subject is determined as being heterozygous for Z-AAT and M-AAT. When the solid support exhibits a detectable signal in the second position of the test zone and not the first position, the subject is determined as being homozygous for M-AAT. In various aspects of the disclosure, the method further comprises treating the subject for AAT deficiency when the subject is determined to be homozygous for Z-AAT or heterozygous for Z-AAT and M-AAT.

### EXAMPLES

### Example 1

This example describes an exemplary method of detecting M-AAT in a sample. Patients undergoing aerosolized AAT therapy have healthy AAT concentrations in varying amounts in the serum/plasma below the nephelometric range (1-5 μM). This Example demonstrates that the materials and methods described herein are capable of distinguishing healthy AAT from the deficient form below this range. The detection of healthy AAT in the serum/plasma of a PI ZZ patient undergoing AAT aerosolization indicates that the treatment is successfully reaching the alveolar space.

In this assay, a monoclonal antibody specific for M-AAT and comprising CDR sequences of SEQ ID NOs: 3-8 was nonspecifically adsorbed to microtiter wells and incubated with plasma or serum. Antibody: antigen complexes were then incubated with a second (detection) antibody, followed by peroxidase conjugated IgG. A colorimetric substrate was added, the amount of which is proportional to the quantity of AAT in the sample.

Repeatability was evaluated by performing independent measurements of three different levels of a purified M-AAT known to be within the linear range of the assay and evaluating the % CV. Accuracy was assessed by determining the mean value of each of the three standards and comparing them to their known theoretical value. The results of the assays performed on 15 pM, 50 pM, and 166.67 pM concentrations of control are summarized as follows. The means of each of the three levels tested were 14.51 pM, 48.41 pM, and 164.56 pM, respectively, corresponding to 96.73%, 96.82%, and 98.73% recovery. The precision of the method was confirmed with the % CV measurements all falling below 10% at 8.34%, 3.82% and 7.89%, respectively.

Reproducibility was evaluated with six assays between two analysts over time. Reagents were changed midway through the testing. The inter-assay precisions (% CV) were 3.82% and 8.34%, demonstrating that the reproducibility of the assay is excellent. Different analysts, run dates, and reagents had no significant impact on the outcome of the assay.

Selectivity was judged by measuring six independent serum/plasma samples both neat and spiked with AAT. Three levels of AAT (12.6 nM, 126 nM and 252 nM) were spiked into six Z plasma samples and percent recovery for each was calculated from the measured versus the theoretical values. At the low and mid-level spike, recovery was successful with a mean accuracy of 95.20% and 101.22% and % CV of 3.53% and 3.90%, respectively. At the high level, five samples had a recovery between 102.66% and 114.20% and the % CV of the values was 4.3%. It was determined that the sixth sample was outside the range likely due to an error in making the initial spike dilution.

The lower and upper limits of quantification were set by determining the lowest and highest concentration of the M-AAT standard that could be accurately measured over six independent repetitions with acceptable precision. Values were not obtainable at 4.335 pM or 6.503 pM. The % CV of the 9.755 pM replicates was 6.97% with an average recovery of 96.02%.

Linearity and range were determined by analyzing five independent curves within the dynamic range of the assay's application. The calibration curves were established using a log-log method and at least six points were required in the middle of the curve for acceptance. The dynamic range for M-AAT measurement by ELISA was established to be between 250 pM and 4.335 pM. Five standard curves were evaluated between these concentrations and the slopes determined to range from 0.94-1.1. Individual point deviations ranged from −17.39%-12.00%.

The Example demonstrates that an M-AAT ELISA procedure for measuring levels of M-AAT in serum/plasma samples using the M-AAT binding protein described herein performed satisfactorily and is capable of producing accurate data when measuring freshly thawed samples (fewer than three freeze-thaw cycles), samples that have been stored at 2-8° C. (for fewer than three days), or samples that have sat at room temperature (for up to four hours).

### Example 2

This Example describes materials and methods for further characterizing the binding proteins described herein.

Alpha-1 antitrypsin (AAT) is the most abundant serine proteinase inhibitor. The M-variant of alpha-1 antitrypsin (M-AAT) is the most common wild type allele/variant of AAT. The Z-variant of alpha-1 antitrypsin (Z-AAT) is the allele most likely to cause both liver and lung disease. The concentrations of four different lots of purified Z Alpha-1 Antitrypsin (Z-AAT) and one lot of purified M Alpha-1 Antitrypsin were quantified by Pierce BCA assay. The purified proteins were then normalized to an equivalent concentration (so as to normalize protein loaded) and run under standard denaturing (SDS-PAGE) and western blotting conditions. Nitrocellulose was selected as the matrix for blotting. Pre-blocking and subsequent antibody incubation steps were carried out in standard blocking buffer consisting of tris-buffered saline with 0.1% tween (TBS-T) supplemented with %5 (w/v) blotting-grade milk. The blot was then probed with mouse monoclonal ∝-M-AAT antibody (MAAT mAb) followed by Polyclonal Goat ∝-mouse IgG (H+L) conjugated to horseradish peroxidase (GAMIG-HRP). Chemiluminescent substrate was then applied to the blot and the resulting image shown above was captured.

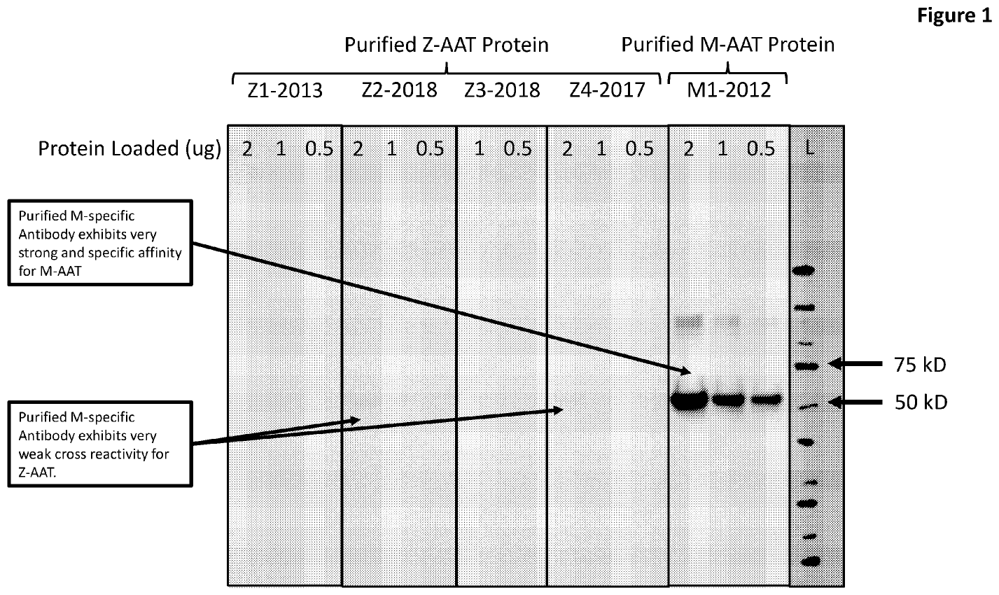

The results are illustrated in FIG. 1, which shows that MAAT mAb detected denatured M-AAT with a high degree of specificity, yielding strong bands associated with M-AAT and essentially undetectable bands where Z-AAT was loaded. This is particularly interesting as the purified Z-AAT lots each contain Z-AAT purified from the plasma of multiple patients (Pi*ZZ) that are homozygous for the Z-allele of SerpinA1, the gene responsible for Z-AAT production. All variants of AAT were detected on the same blot when probed with rabbit polyclonal anti-total-AAT antibody that reacts with both M-AAT and Z-AAT.

Specific binding of Z-specific antibodies also was determined. Purified Z Alpha-1 Antitrypsin (Z-AAT) and purified M Alpha-1 Antitrypsin previously quantified by Pierce BCA assay were normalized to an equivalent concentration (so as to normalize protein loaded) and run under standard denaturing (SDS-PAGE) and western blotting conditions. Nitrocellulose was selected as the matrix for blotting. The blot was subdivided into multiple strips, each one containing 2 μg of Z-AAT, 2 μg of M-AAT, and Precision Plus Protein Standards. Pre-blocking and subsequent antibody incubation steps were carried out in modified blocking buffer consisting of tris-buffered saline (TBS) with %5 (w/v) blotting-grade milk. Each strip of the blot was then probed with hybridoma supernatant containing mouse monoclonal ∝-Z-AAT antibody (ZAAT mAb) followed by Polyclonal Goat anti-mouse IgG (H+L) conjugated to horseradish peroxidase (GAMIG-HRP). Chemiluminescent substrate was then applied to the blot and the resulting image shown above was produced by combining the images captured from the development of each blot/strip. Blocking buffer for the blocking and antibody steps required the absence of Tween 20. Detection of Z-AAT on the blot failed when Tween 20 was included in wash buffers and blocking buffers. Wash buffer consisted of PBS with no Tween added.

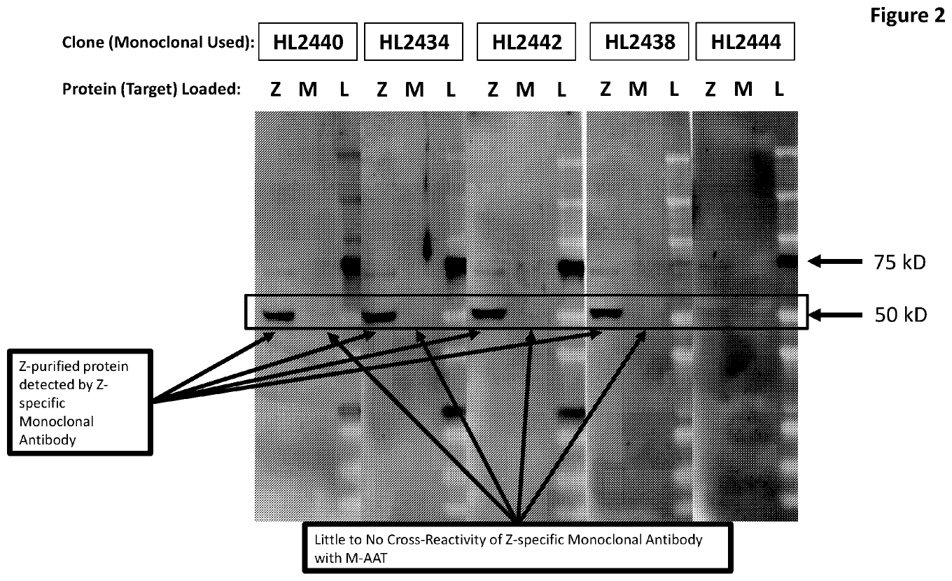

The results of the study are illustrated in FIG. 2 and show that Z-AAT mAb (unpurified—hybridoma supernatant) detected denatured Z-AAT (immobilized onto a nitrocellulose membrane by Western Blot) with a high degree of specificity, yielding strong bands associated with Z-AAT and essentially undetectable bands where M-AAT was loaded.

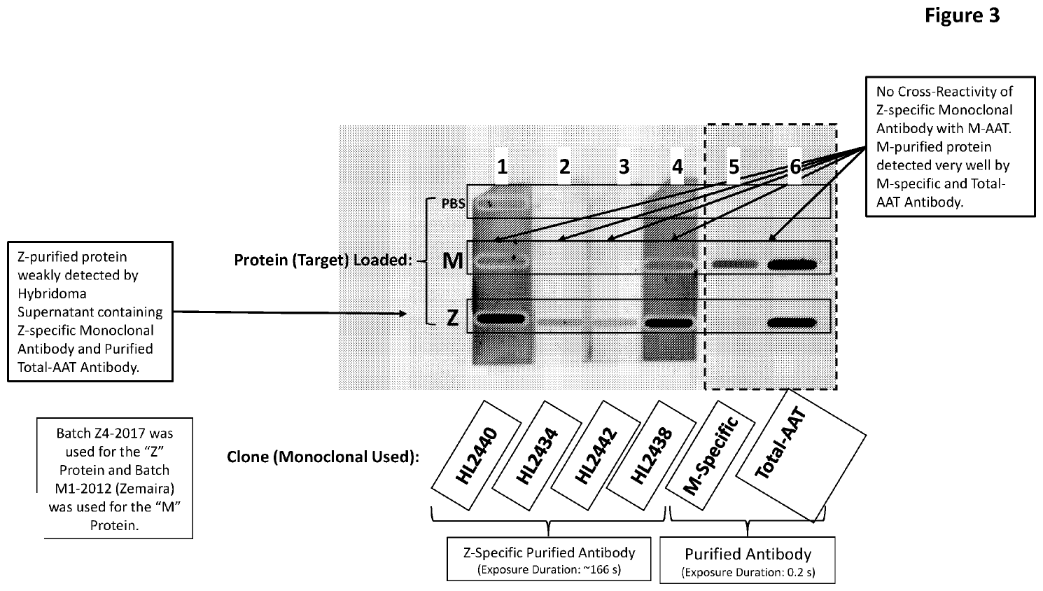

Antibody specificity was further examined using dot blots. See FIG. 3. Normalized amounts (3 μg) of purified Z Alpha-1 Antitrypsin (Z-AAT) and purified M Alpha-1 Antitrypsin were loaded onto nitrocellulose via vacuum-assisted dot-blot apparatus. The blot was subdivided into multiple strips, each one containing three wells corresponding to a blank well with only phosphate-buffered saline (PBS), one well with 3 μg of M-AAT, and another with 3 μg of Z-AAT. In FIG. 3, the first four strips from left to right were treated accordingly: each strip of the blot was then probed with one of four hybridoma supernatants containing mouse monoclonal ∝-Z-AAT antibody (Z-AAT mAb). The fifth strip from the left was probed with purified mouse monoclonal anti-M-AAT antibody (M-AAT mAb), and the sixth strip from the left was probed with rabbit polyclonal anti-Total-AAT. For strips 1-5 from the left, this step was followed by the addition of polyclonal goat anti-mouse IgG (H+L) conjugated to horseradish peroxidase (GAMIG-HRP) for detection of bound mouse monoclonal anti-Z-AAT antibody (Z-AAT mAb) or mouse monoclonal anti-M-AAT antibody (M-AAT mAb). For the 6th strip from the left, detection was achieved by the addition of polyclonal goat anti-rabbit IgG (H+L) conjugated to horseradish peroxidase (GARIG-HRP). Chemiluminescent substrate was then applied to the blot and the resulting image shown above was produced by combining the images captured from the development of each blot/strip. Blocking buffer for the blocking and antibody steps required the absence of Tween 20. Detection of Z-AAT by Z-AAT mAb on the blot failed when Tween 20 was included in wash buffers and blocking buffers. Blocking and wash buffers for the first four strips utilized HEPES-buffered saline without Tween 20 at all steps including antibody and antigen incubation steps. Blocking and wash buffers for the fifth and sixth strips utilized TBS-T (with Tween 20) at all steps including antibody and antigen incubation steps. Blocking buffer, regardless of whether HEPES-based or TBS-based, contained %5 (w/v) blotting grade milk.

The results provided in FIG. 3 show that unpurified Z-AAT mAb originating from hybridoma supernatant detected native Z-AAT adhered via vacuum assisted dot-blot. The detection occurred with a high degree of specificity and low degree of sensitivity which can most likely be attributed to the use of hybridoma supernatant. M-AAT mAb provided highly specific and highly sensitive results utilizing standard blocking and wash buffers containing Tween 20. Anti-Total-AAT showed that both M-AAT and Z-AAT purified protein were successfully adhered and ultimately detected by this assay.

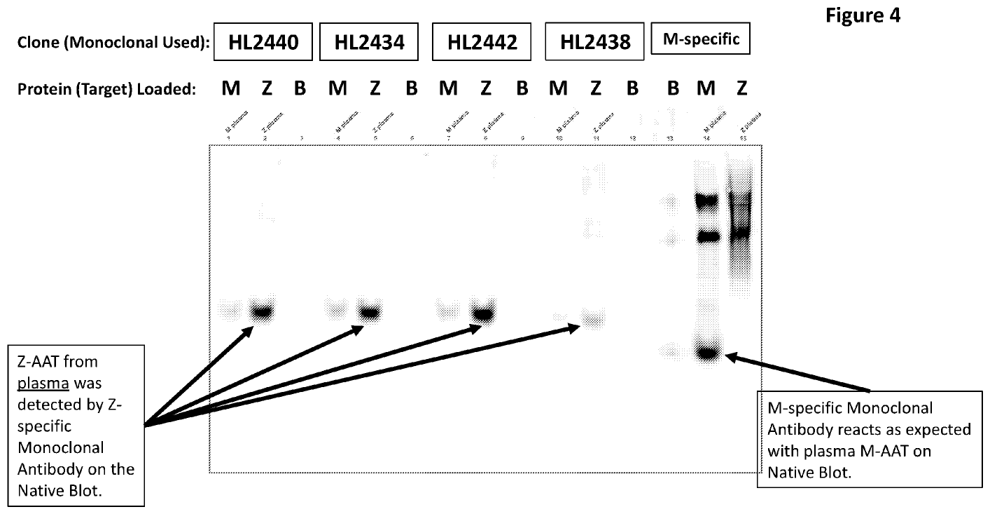

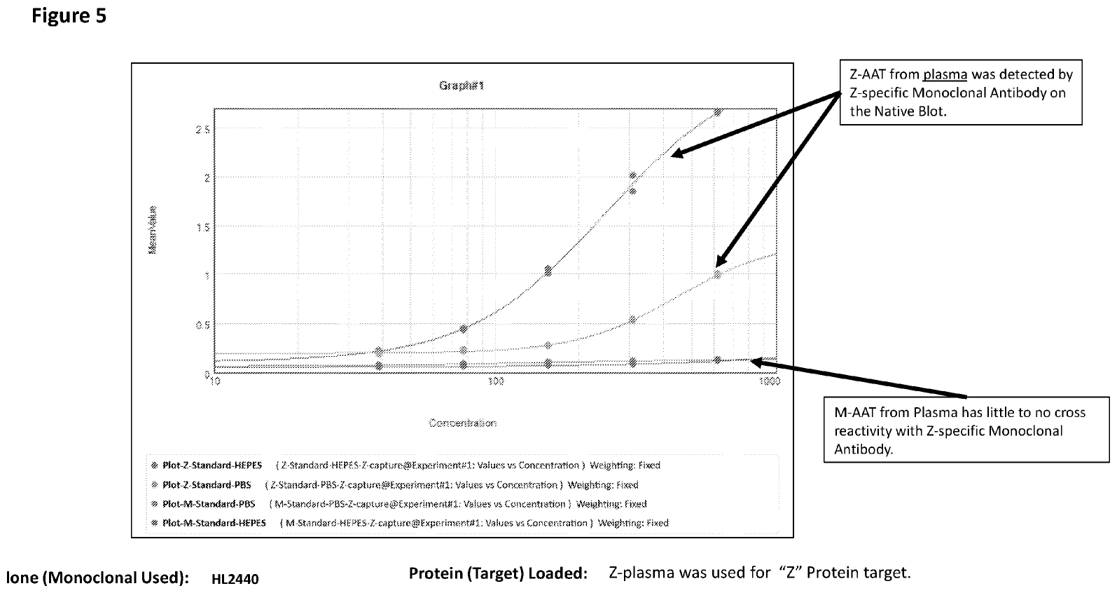

Cross-reactivity of the Z-and M-binding proteins of the disclosure was further examined. Plasma concentrations of AAT from a homozygous ZZ-allele patient and a homozygous MM-allele individual were determined by nephelometry. Plasma was then diluted and normalized so that equal amounts (0.161 μg) of M Alpha-1 Antitrypsin and Z Alpha-1 Antitrypsin (Z-AAT) in plasma were loaded into each lane of the gel. In order from left to right in FIG. 4, the blot was subdivided into multiple strips, each one containing 3 wells corresponding to a blank well with only PBS (“B” in FIG. 4), one well with 0.161 μg of M-AAT (“M” in FIG. 4), and another with 0.161 μg of Z-AAT (“Z” in FIG. 4). Standard, native (non-denaturing) conditions for poly-acrylamide gel electrophoresis were used, followed by blotting in the absence of methanol or ethanol to preserve native confirmation of target proteins. The nitrocellulose membrane/blot was then cut into six strips. The first four strips from left to right were treated as follows: each strip of the blot was probed with one of four hybridoma supernatants containing mouse monoclonal anti-Z-AAT antibody (ZAAT mAb). The fifth strip was probed with purified mouse monoclonal anti-M-AAT antibody (MAAT mAb). A sixth strip was probed with rabbit polyclonal anti-Total-AAT (not shown). Polyclonal goat anti-mouse IgG (H+L) conjugated to horseradish peroxidase (GAMIG-HRP) was added for detection of bound mouse monoclonal anti-Z-AAT antibody (ZAAT mAb) or mouse monoclonal anti-M-AAT antibody (MAAT mAb). The strip probed with rabbit polyclonal anti-Total-AAT was exposed to polyclonal goat anti-rabbit IgG (H+L) conjugated to horseradish peroxidase (GARIG-HRP). Chemiluminescent substrate was then applied to the blots and the resulting image shown in FIG. 4 was produced by combining the images captured from the development of each blot/strip. HEPES-based blocking and wash buffers for the blocking and antibody steps required the absence of Tween 20. Detection of Z-AAT on the blot failed when Tween 20 was included in wash buffers and blocking buffers (result not shown), and as a result, Tween 20 was excluded from buffers when working with ZAAT mAbs.

The results (illustrated in FIG. 4) show that ZAAT mAb (HL2440, HL2434, HL2442, and HL2438) detected denatured Z-AAT with a high degree of specificity, yielding strong bands associated with Z-AAT and essentially undetectable bands where M-AAT was loaded. M-AAT mAb showed limited cross-reactivity with Z-AAT but cross-reactivity was certainly higher than when TBS based blocking and wash buffers containing Tween 20 were utilized. Banding pattern differences between monomeric Z-AAT and M-AAT are evident on this gel and are due to variations in the isoelectric point of these variants of AAT when run on a native gel where proteins have not been reduced.

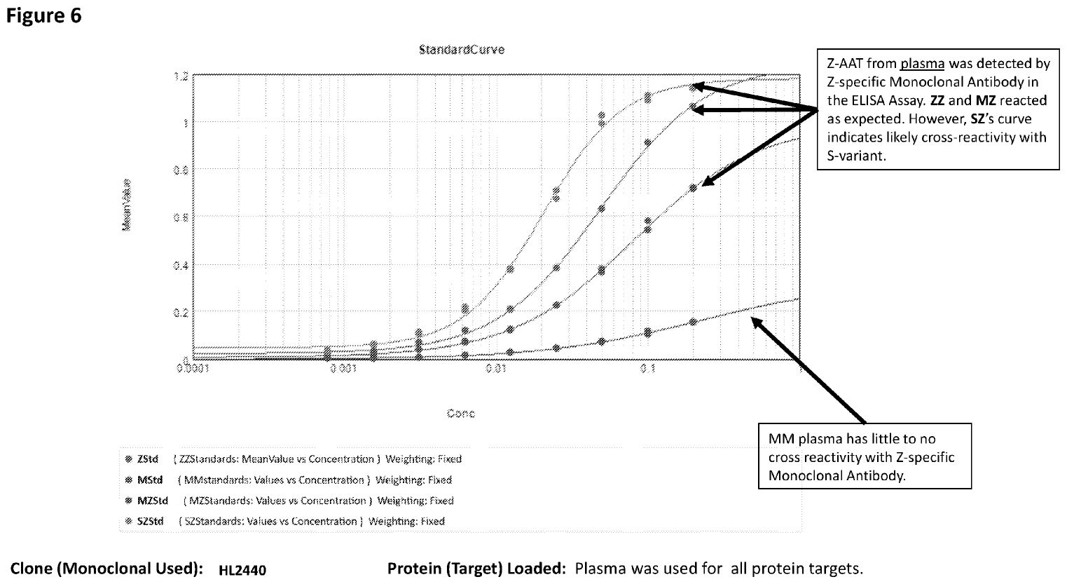

FIG. 5 provides a summary of the cross-reactivity of ZAAT mAb when used as the coating/capture antibody and exposed to plasma from varying AAT phenotypes/serotypes, including S/S, Ma/Ma, and M/M serotypes. The values provided are relative values calculated by assuming the absorbance value for Z/Z plasma under each condition outlined represents 100% on-target signal. Thus, using the conditions associated with the pH 7.4 assay as an example, when plasmas of multiple serotypes at a set dilution of 1/40 were incubated in HEPES buffered saline at pH 7.4, the absorbance value of M/M plasma was roughly 111×s weaker than the absorbance value of Z/Z plasma. The results show that specificity for Z/Z relative to other serotypes is best under physiological (pH 7.4) conditions. The standard curves that coincide with these results are found in FIG. 6, which shows fluctuations in the dynamic response of antigen/antibody binding across the variable pH and wash conditions. The “association step” refers to the step when ZAAT mAb bound to the ELISA plate is incubated with multiple dilutions of Z/Z plasma where the concentration of ZAAT has been determined with nephelometry prior to use in this assay. M/M plasma at a 1/40 dilution in HEPES-buffered saline has a 4× higher concentration of alpha-1 antitrypsin than that found in Z/Z plasma and yet is only weakly detected by an absorbance value that is 111×s less intense than the Z/Z plasma dilution at 1/40.

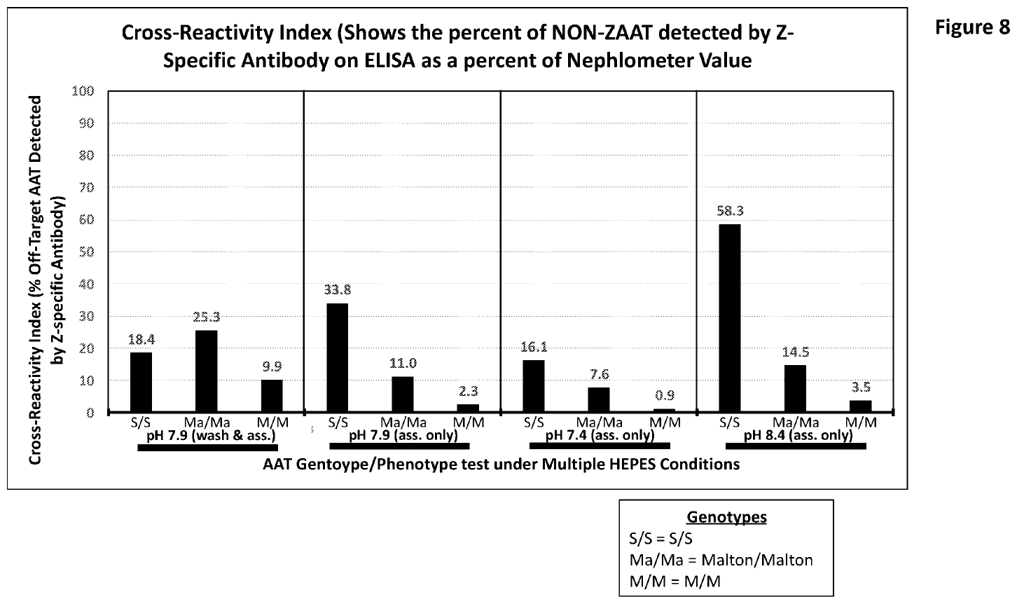

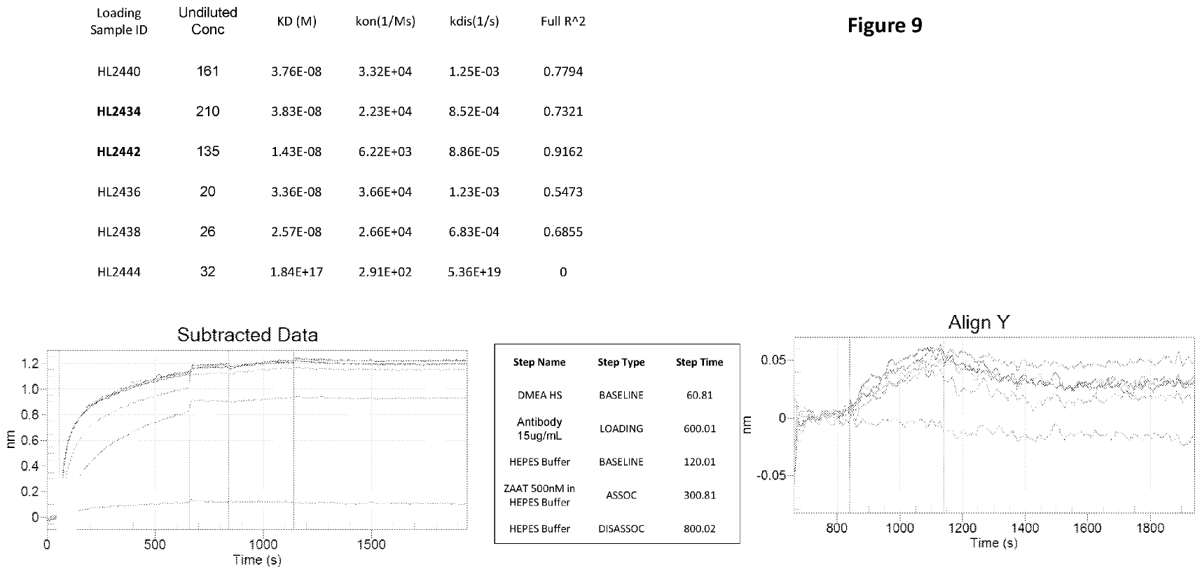

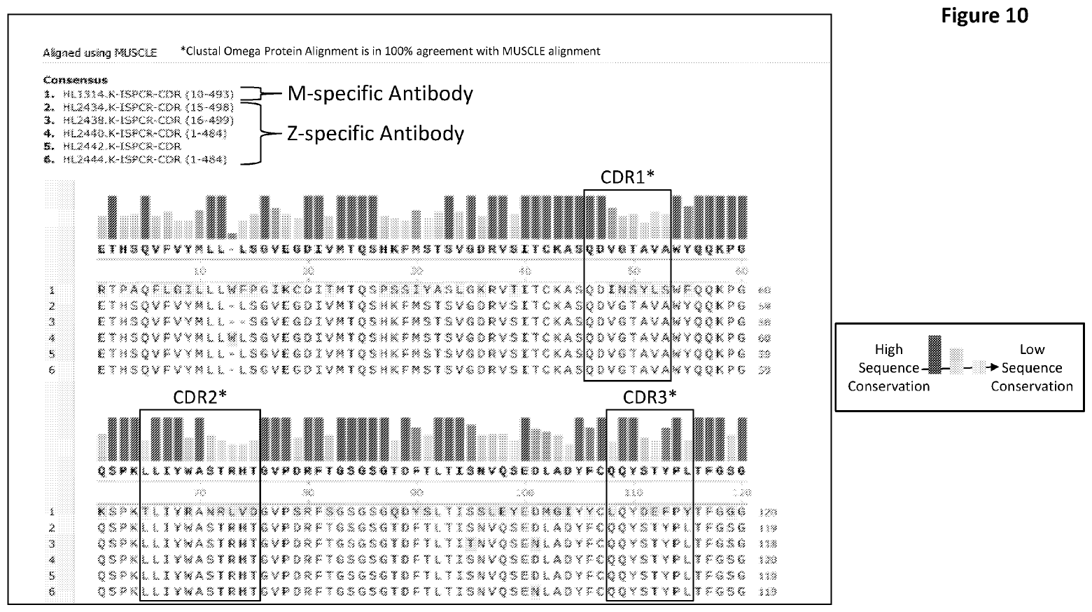

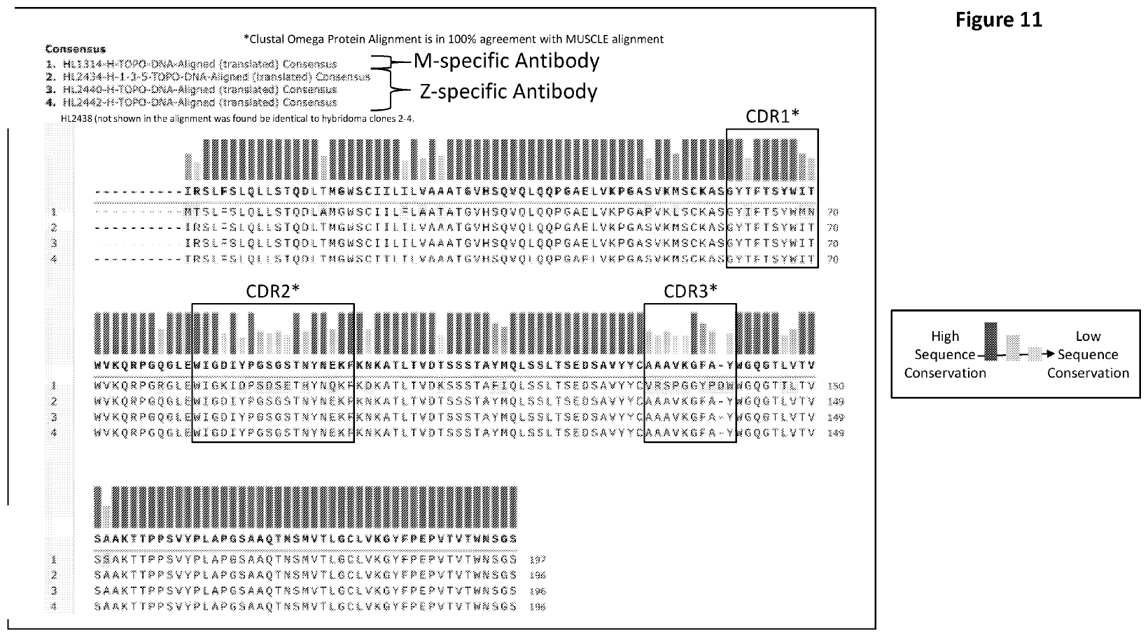

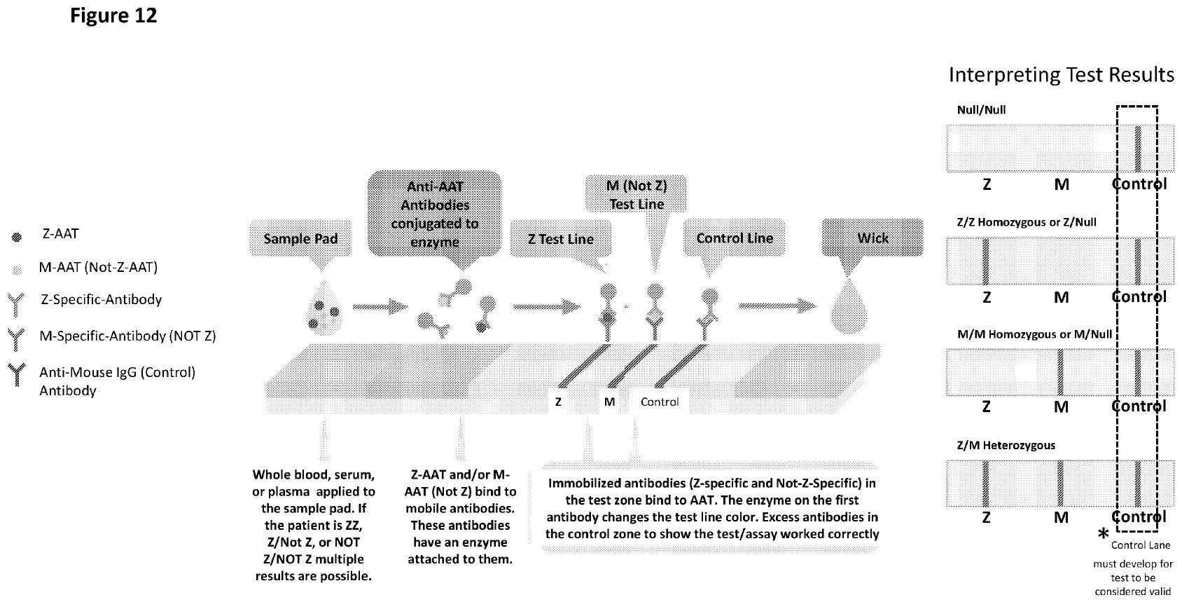

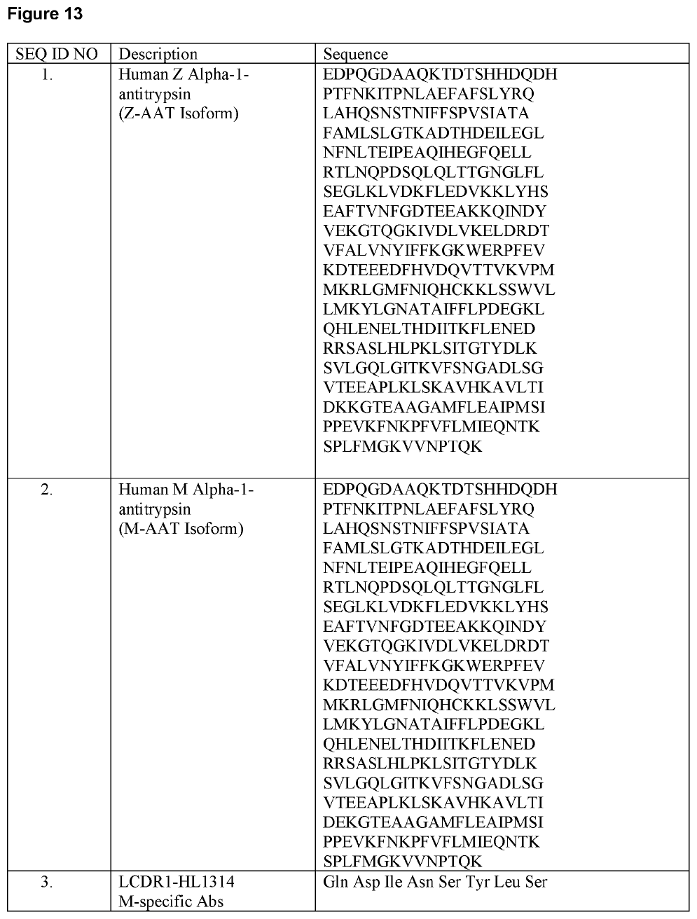

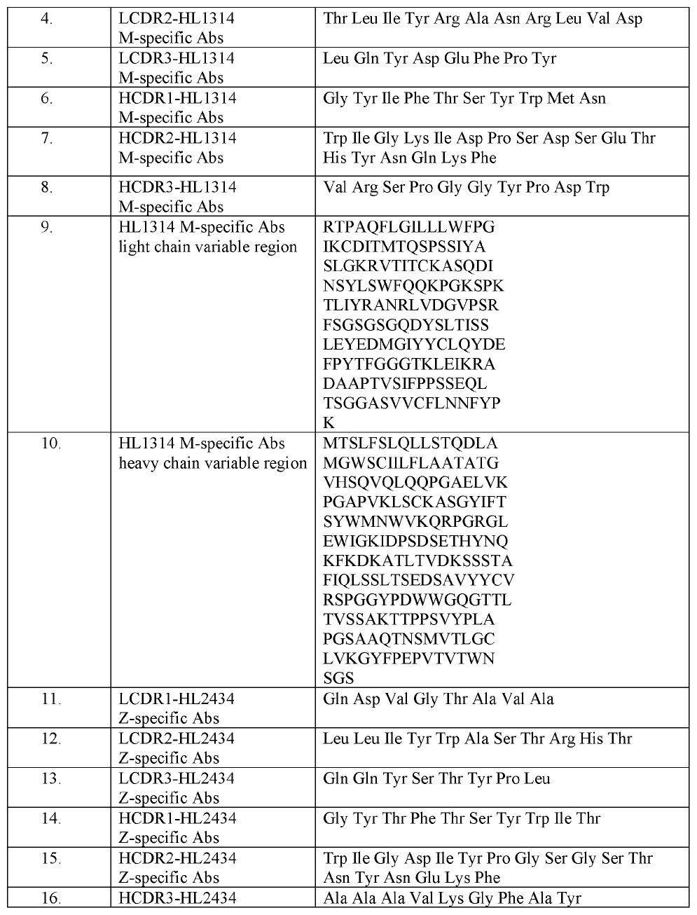

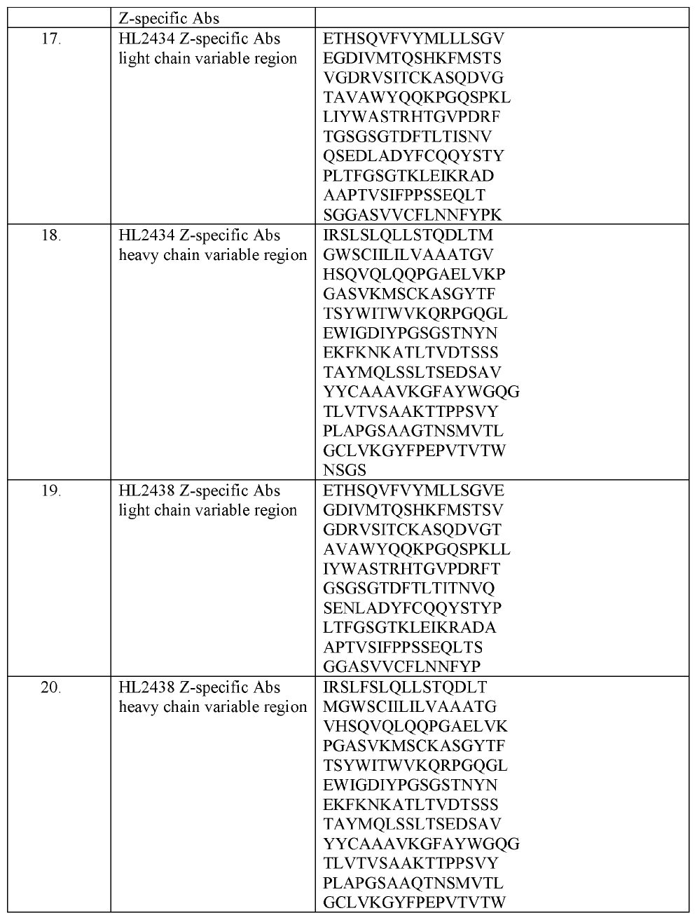

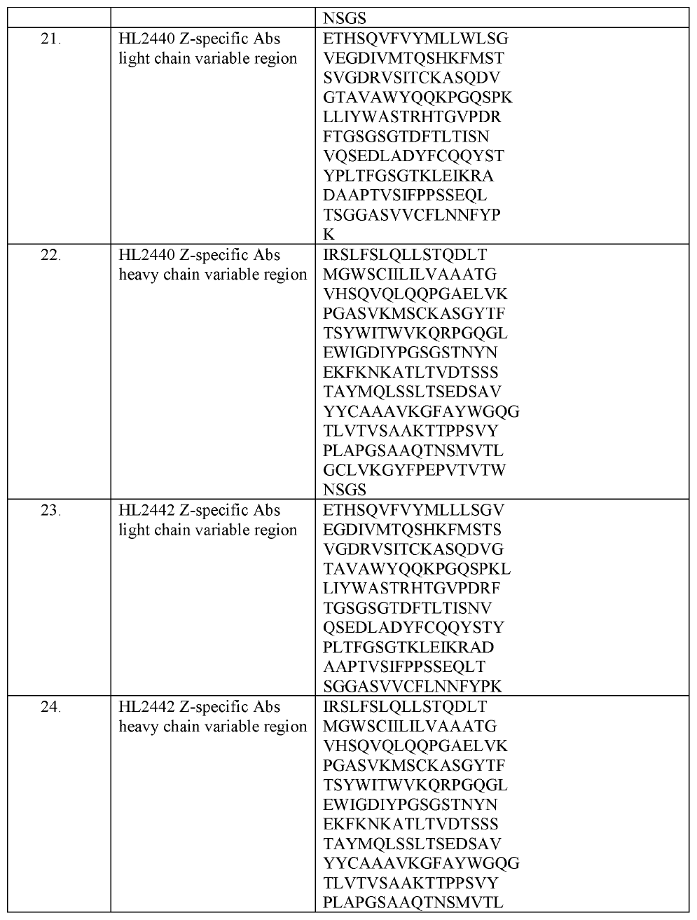

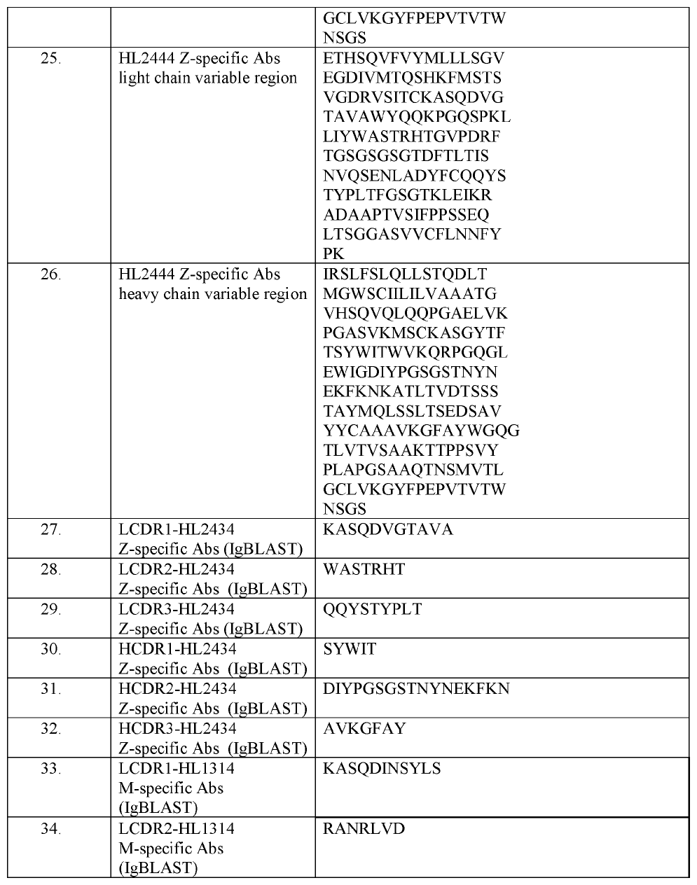

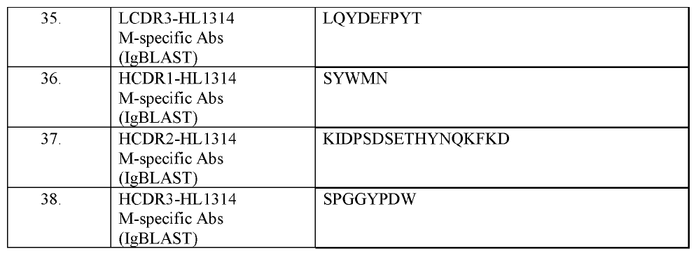

The assay behind FIG. 6 was performed as follows. Purified ZAAT mAb was coated onto a high-binding ELISA plate using Voller's Buffer. The plate was then washed to remove unbound coating antibody. Three out of four standard curves used PBS with Tween 20 (PBS-T) to wash away remaining unbound ZAAT mAb and HEPES-buffered saline at varying pHs for the association step, whereas one plate utilized both HEPES-buffered saline at pH 7.9 (no Tween added) for both the post-coating wash step and antigen/antibody association step so that the ZAAT mAb never came into contact with Tween 20 prior to incubation with antigen (Z-AAT). Following the association step where antigen in one of four blocking buffers (as indicated above) was incubated in the presence of coated ZAAT mAb, subsequent wash and detection steps were performed using PBS-T based blocking buffer supplemented with %5 (w/v) blotting-grade milk and PBS-T (wash buffer). The association step was followed by the addition of rabbit polyclonal anti-Total-AAT to enable facilitate detection of Z-AAT bound to the capture antibody. Following several washes, polyclonal goat anti-rabbit IgG (H+L) conjugated to horseradish peroxidase (GARIG-HRP) was added to each well of the ELISA plate to allow for detection of rabbit polyclonal anti-Total-AAT. OPD colorimetric (HRP-specific) substrate was then applied to all wells and allowed to develop for 30 minutes. The results of that development and the corresponding standard curves are shown. These standard curves were used alongside known dilutions of plasma from Pi*ZZ, Pi*MM, Pi*SS, Pi*Malton/Malton patients to evaluate cross reactivity of ZAAT mAb with those variants of AAT. Those results are summarized on FIG. 8.

The results from previous ELISAs showed that ZAAT mAb detected denatured Z-AAT with a high degree of specificity, especially when compared with M-AAT, which is the predominant phenotype. The results of FIG. 6 show that binding of ZAAT in plasma can be significantly enhanced by using HEPES-buffered saline and HEPES based blocking buffers that omit Tween 20. Results appear best when all wash steps prior to and including the association step omit the use of Tween 20. The curves highlight the improved dynamic range of the standard curve when physiological pH of 7.4 is used.

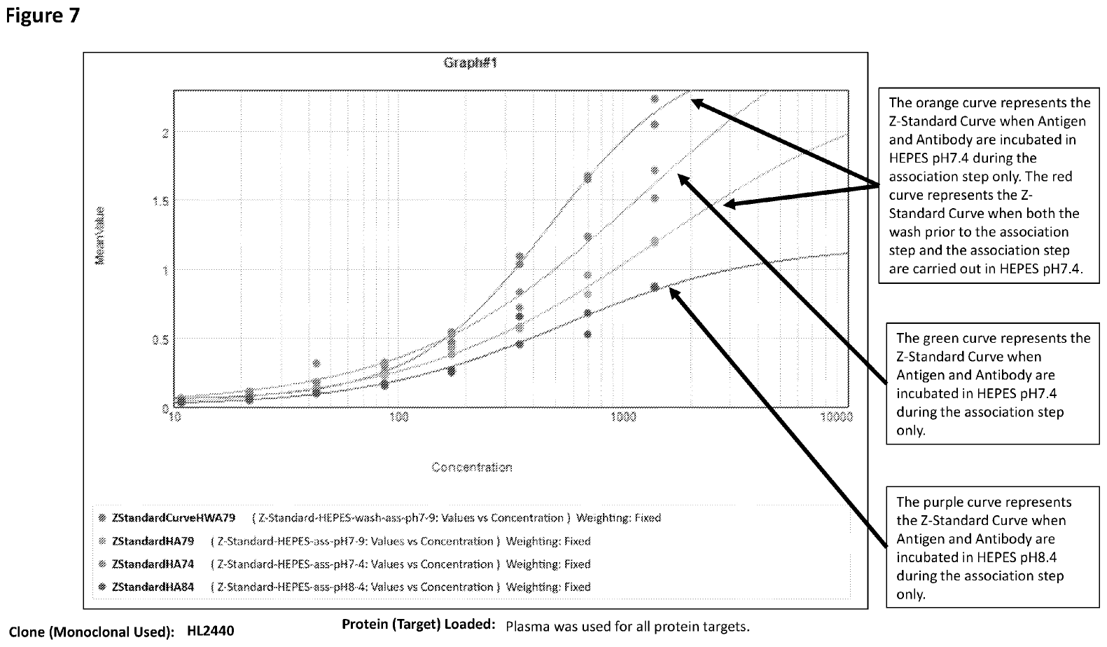

The results illustrated in FIG. 7 show antibody/antigen binding kinetics when using the Octet system. The system relies upon the principle that changes in the number of molecules bound to the biosensor causes a shift in the interference pattern that is measured in real time and plotted. Six hybridoma clones representing theoretically unique hybridoma clones were screened using the Octet system to determine biomolecular binding kinetics. DMEA growth media containing horse serum was used to establish a baseline reading on the Octet. Next, crude antibody (hybridoma supernatant) at 15 μg/mL was loaded in the “loading step.” HEPES buffered saline was then used to establish a second baseline following coating of the biosensor with antibody. Biosensor coated with antibody (ligand) was then incubated with the targeted antigen (purified Z-AAT protein) during the association step. The kinetic ON rate is described above and the results calculated for each antibody are shown in the table at the top of the figure. The dissociation step then involved placing the biosensor into HEPES buffer with no targeted antigen so that the kinetic OFF rate could be measured, in which the targeted antigen can spontaneously dissociate from the antibody bound to the biosensor. Based on the kON and kOFF rates of the tested unpurified antibodies, several stand out as offering conducive kinetics for use in assays. The performance of purified and unpurified ZAAT mAb (HL2434, HL2438, HL2440, and HL2442) in HEPES and PBS based buffers was confirmed.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. It should be understood that while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” the feature.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A binding protein that binds human Z Alpha-1 Antitrypsin (Z-AAT) and comprises: (a) an HCDR1 comprising the amino acid sequence of SEQ ID NO: 14; (b) an HCDR2 comprising the amino acid sequence of SEQ ID NO: 15; (c) an HCDR3 comprising the amino acid sequence of SEQ ID NO: 16; (d) an LCDR1 comprising the amino acid sequence of SEQ ID NO: 11; (e) an LCDR2 comprising the amino acid sequence of SEQ ID NO: 12; and (f) an LCDR3 comprising the amino acid sequence of SEQ ID NO 13.

2. A binding protein that binds human Z Alpha-1 Antitrypsin (Z-AAT) and comprises CDRs set forth in a light chain variable region having the amino acid sequence of SEQ ID NO: 17, 19, 21, 23, or 25 and CDRs set forth in a heavy chain variable region having the amino acid sequence of SEQ ID NO: 18, 20, 22, 24, or 26

3. The binding agent of claim 1, comprising a light chain variable region comprising the amino acid sequence of SEQ ID NO: 17, 19, 21, 23, or 25.

4. The binding agent of claim 1, comprising a heavy chain variable region comprising the amino acid sequence of SEQ ID NO: 18, 20, 22, 24, or 26.

5.-11. (canceled)

12. A binding protein that binds human M Alpha-1 Antitrypsin (M-AAT) and comprises: (a) an HCDR1 comprising the amino acid sequence of SEQ ID NO: 6; (b) an HCDR2 comprising the amino acid sequence of SEQ ID NO: 7; (c) an HCDR3 comprising the amino acid sequence of SEQ ID NO: 8; (d) an LCDR1 comprising the amino acid sequence of SEQ ID NO: 3; (e) an LCDR2 comprising the amino acid sequence of SEQ ID NO: 4; and (f) an LCDR3 comprising the amino acid sequence of SEQ ID NO: 5.

13. A binding protein that binds human M Alpha-1 Antitrypsin (M-AAT) and comprises CDRs set forth in a light chain variable region comprising the amino acid sequence of SEQ ID NO: 9 and CDRs set forth in a heavy chain variable region comprising the amino acid sequence of SEQ ID NO: 10.

14. The binding agent of claim 12, comprising a light chain variable region comprising the amino acid sequence of SEQ ID NO: 9.

15. The binding agent of claim 12, comprising a heavy chain variable region comprising the amino acid sequence of SEQ ID NO: 10.

16.-22. (canceled)

23. A method for detecting Z-alpha-1 antitrypsin (Z-AAT) in a subject, the method comprising:
contacting a biological sample from a subject with the binding protein of claim 1 and
detecting binding of the binding protein to Z-AAT in the sample.

24. A method of characterizing delivery of M-AAT to a subject suffering from AAT deficiency, the method comprising:
contacting a biological sample from a subject homozygous for Z-AAT and previously administered aerosolized M-AAT with the binding protein of claim 12, and
detecting binding of the binding protein to M-AAT in the sample, wherein binding indicates that the aerosolized M-AAT has reached the alveolar space of the subject.

25. A solid support comprising the binding protein of claim 12.

26. A solid support comprising the binding protein of claim 1.

27. The solid support of claim 25, wherein the solid support is a dipstick, test strip, or micro-well plate.

28. The solid support of claim 26, wherein the Z-AAT binding protein is immobilized at a first position on the surface of a solid support and the solid support further comprises a binding protein that binds M-AAT immobilized at a second position on the surface of the solid support.

29. The solid support of claim 28, wherein the binding protein that binds M-AAT is the M-AAT binding protein of claim 12.

30. The solid support of claim 28 further comprising an anti-AAT detection antibody that binds both M-AAT and Z-AAT and which is bound to a detection agent.

31. The solid support of claim 30 further comprising a capture antibody which binds the anti-AAT detection antibody.

32. The solid support of claim 31, wherein the solid support comprises
a sample application zone;
a detection antibody zone adjacent to the sample application zone, wherein anti-AAT detection antibodies are adhered to the solid support such that the anti-AAT detection antibodies are released upon contact with a biological sample and/or buffer;
a test zone adjacent to the detection antibody zone, wherein the test zone comprises the Z-AAT binding protein in a first position and an M-AAT binding protein comprising (a) an HCDR1 comprising the amino acid sequence of SEQ ID NO: 6; (b) an HCDR2 comprising the amino acid sequence of SEQ ID NO: 7; (c) an HCDR3 comprising the amino acid sequence of SEQ ID NO: 8; (d) an LCDR1 comprising the amino acid sequence of SEQ ID NO: 3; (e) an LCDR2 comprising the amino acid sequence of SEQ ID NO: 4; and (f) an LCDR3 comprising the amino acid sequence of SEQ ID NO: 5 in a second position;
a control zone adjacent to the test zone, wherein the control zone comprises capture antibodies which bind the detection antibody immobilized on the solid support, and a wick.

33. A kit comprising the solid support of claim 25 with instructions for use.

34. A method of detecting AAT deficiency in a subject, comprising adding a sample obtained from a subject to the solid support of claim 32, wherein, when the solid support exhibits a detectable signal in the first position of the test zone and not the second position, the subject is determined as being homozygous for Z-AAT; when the solid support exhibits a detectable signal in each of the first position and the second position, the subject is determined as being heterozygous for Z-AAT and M-AAT; and when the solid support exhibits a detectable signal in the second position of the test zone and not the first position, the subject is determined as being homozygous for M-AAT.
